(19) United States
(12) Patent Application Publication
(10) Pub. No.: US 2012/0329158 A1
(43) Pub. Date: Dec. 27, 2012

(54) Methods of Plant Regeneration and Apparatus Therefor

(76) Inventors: Prakash Lakshmanan, Queensland (AU); Angela Mordocco, Queensland (AU)

(21) Appl. No.: 13/522,090

(22) PCT Filed: Jan. 13, 2011
(86) PCT No.: PCT/AU2011/000034

§ 371 Date: Aug. 28, 2012

(30) Foreign Application Priority Data
Jan. 13, 2010 (AU) 2010900137

(51) Int. Cl. A01H 4/00; A01C 1/06; C12N 5/04

(52) U.S. Cl. 435/430; 435/420; 47/57.6

(57) ABSTRACT

A method of preparation of a plant tissue fragment is provided wherein apical dominance of plant meristematlc tissue is inhibited followed by fragmentation of the tissue. Also provided are methods of plant micropropagation and methods of artificial seed production using apical dominance suppression in preferably, a semi-automated process. Also provided is a plant tissue processing machine that generates plant fragments with high regeneration efficiency and an artificial seed production apparatus.

### FIELD OF THE INVENTION

THIS invention relates to plant regeneration. More particularly, this invention relates to apparatus' and methods regenerating plants in a high-throughput manner under septic conditions.

### BACKGROUND TO THE INVENTION

These have been many efforts in automating various steps of micropropagation and artificial plant seed production technology. These include the concepts and practical demonstration of temporary immersion systems, various forms of bioreactor technologies adapted to micropropagation, attempts of generating tissue cutting technologies such as robots and photoautotrophic culture systems. All these were aimed at reducing labour cost to make large-scale commercial micropropagation more efficient and economically competitive.

Plant regeneration using artificial plant seed technology is an alternative to traditional micropropagation for production and delivery of cloned plantlets. Several aspects of this technology remain underdeveloped for large scale commercialisation use. Much of the work using artificial seed technology has focused on somatic embryos as the tissue of choice. For many plant species, somatic embryogenesis, the process of producing somatic embryos, is often long, labour-intensive, genotype-specific and may lead to genetic or phenotypic changes. Hence, artificial seeds have been derived from non-embryogenic tissue but there remains an undesirable economy of commercial production particularly in terms of labour costs.

Despite progress being made with respect to artificial seed technology, efficient production of mature monocotyledous plants displaying minimal somoclonal variation has remained elusive. Weyerhaeuser has developed an automated somatic embryogenesis, embryo sorting and embryo encapsulation technology for pines that is commercially used. Somaclonal variants often result in reduced agronomic performance compared with the plant(s) from which they are derived. Somaclonal variation is particularly evident with callus-based regeneration techniques, including somatic embryogenesis, which are used in plant regeneration systems.

### SUMMARY OF THE INVENTION

Despite progress having been made in micropropagation and in particular, artificial plant seed development, widespread commercial use is relatively limited due to, in part, high labour costs and the physical constraints on scale-up.

Therefore the invention is broadly directed to apparatus and methods suitable for use in plant micropropagation and more particularly, regenerating propagules aseptically in a high-throughput manner.

In other broad aspects, the invention is directed to a plant tissue processing apparatus that generates plant tissue fragments that do not require a developmental stage in culture media prior to artificial plant seed production.

In other broad aspects, the invention provides methods and systems that are at least partially automated, semi-automated or fully automated.

In a first aspect, the invention provides a method of preparing a plant meristematic tissue fragment for use in plant micropropagation, said method including the steps of:
(i) inhibiting apical dominance of a plant meristematic tissue; and
(ii) fragmenting the plant meristematic tissue resulting from step (i) to prepare the plant meristematic tissue fragment far use in plant micropropagation.

In a second aspect, the invention provides a plant meristematic tissue fragment produced according to the method of the first aspect.

In a third aspect, the invention provides a method of plant micropropagation, said method including the steps of:
(i) inhibiting apical dominance of a plant meristematic tissue;
(ii) fragmenting the plant meristematic tissue resulting from step (i) to thereby produce a plant meristematic tissue fragment; and
(iii) regenerating a plant or a plant tissue from the plant meristematic tissue fragment.

In a fourth aspect, the invention provides a method of producing an artificial plant seed, said method including the steps of:
(i) inhibiting apical dominance of a plant meristematic tissue;
(ii) fragmenting the plant meristematic tissue resulting from step (i) to thereby produce a plant meristematic tissue fragment; and
(iii) coating the plant meristematic tissue fragment with a plant tissue-coating medium to thereby produce the artificial plant seed.

In a fifth aspect, the invention provides an artificial seed produced according to the method of the fourth aspect.

In preferred embodiments of any one of the aforementioned aspects, step (i) further includes culturing the plant meristematic tissue whilst maintaining inhibition of apical dominance.

In other preferred embodiments of any one of the aforementioned aspects, the plant meristematic tissue is cultured prior to inhibition of apical dominance.

Preferably, the plant meristematic tissue is cultured for about 4 weeks whilst maintaining inhibition of apical dominance.

In preferred embodiments, inhibiting apical dominance is by way of treatment selected from the group consisting of physical treatment, chemical treatment and biochemical treatment of the plant meristematic tissue.

Preferably, inhibiting apical dominance is by way of physical treatment and more preferrably cutting the plant meristematic tissue, and even more preferably, the plant meristematic tissue is out along a longitudinal axis.

In preferred embodiments of any one of the aforementioned aspects, the plant meristematic tissue is derived from shoot apex.

In preferred embodiments of any one of the aforementioned aspects, the plant meristematic tissue is derived from shoot apical meristem or axillary meristem.

In certain preferred embodiments, step (ii) and/or step (iii) in the method of any one of the aforementioned aspects is preferably at least partially automated, more preferably semi-automated and even more preferably, fully automated.

In a sixth aspect, the invention provides a plant tissue processing apparatus suitable for generating plant tissue fragments for use in plant micropropagation, wherein said plant tissue processing apparatus comprises a plurality of blades wherein at least two (2) blades sever a plant tissue in an ordered sequence along at least two (2) different planes.

Preferably, the plant tissue processing apparatus comprises at least three (3) blades that sever a plant tissue in an ordered sequence along at least three (3) different planes.

In preferred embodiments, the plant micropropagation technique is selected from conventional plant micropropagation and artificial plant seed production.

More preferably, plant micropropagation is artificial plant seed production.

In preferred embodiments, the plant tissue is selected from the group consisting of an axillary bud, a leaf, inflorescence and a shoot apex.

Preferably, the shoot apex tissue is an apical bud tissue and/or an apical meristem tissue.

In a seventh aspect, the invention provides a method of preparing a plant tissue fragment for use in plant micropropagation, said method including the step of (i) cutting a plant tissue using a plant tissue processing apparatus of the sixth aspect, to thereby generate the plant tissue fragment suitable for use in plant micropropagation.

In an eighth aspect, the invention provides a method of producing an artificial plant seed, said method including the step of (i) cutting a plan tissue using a plant tissue processing apparatus of the sixth aspect to thereby generate a plant tissue fragment suitable for use in an artificial plant seed.

In preferred embodiments of any one of the sixth to eighth aspects, the plant tissue is derived from a micro-shoot cluster.

Preferably, the plant tissue and/or micro-shoot cluster is derived from plant tissue selected from the group consisting of an axillary bud, a leaf, inflorescence and a shoot apex.

More preferably, the shoot apex is an apical bud tissue and/or an apical meristem tissue.

In preferred embodiments of the seventh and eighth aspects, the plant tissue is cultured in vitro prior to step (i).

In preferred embodiments of the eighth aspect, the method further includes the step of (ii) coating the plant tissue figment derived from step (i) with a plant tissue-coating medium.

In a ninth aspect, the invention provides a plant tissue fragment produced according to a method of the seventh aspect.

In a tenth aspect, the invention provides an artificial plant seed produced according to a method of the eighth aspect.

In an eleventh aspect, the invention provides an artificial plant seed production apparatus comprising at least two (2) chambers, wherein
a first chamber adapted to contain a plant tissue-coating medium comprising one or more plant tissue fragments; and
a second chamber adapted to contain a seed-coat setting solution,

wherein the first chamber and the second chamber are operatively associated such that discharge of the plant tissue-coating medium from the first chamber into the second chamber thereby forms an artificial plant seed.

In a twelfth aspect, the invention provides a method of plant micropropagation, said method including the step of (i) cutting a plant tissue using a plant tissue processing apparatus of the sixth aspect, to thereby generate the plant tissue fragment suitable for use in plant micropropagation.

In a thirteenth aspect, the invention provides a system for plant micropropagation, said system including a device for fragmenting a plant meristematic tissue with apical dominance inhibited to produce a plant meristematic tissue fragment and either regenerating a plant or a plant tissue from the plant meristematic tissue fragment or coating the plant meristematic tissue fragment with a plant tissue-coating medium.

In preferred embodiments, the system includes one or more elements selected from feature, 3 to 6 of FIG. 37.

In preferred embodiment of any one of the aspects, the micropropragule and/or the artificial plant seed generates monocotyledonous plant or dicotyledonous plant.

More preferably, the monocotyledonous plant is one or more members of the Poaceae family, and more preferably selected from the group consisting of sugarcane, sorghum and wheat and even more preferably, is sugarcane. In other preferred embodiments, the monocotyledonous plant is one or more members of the Musa family and preferably, banana. In yet other preferred embodiments, the monocotyledonous plant is one or more members of the Zingiberaceae family and more preferably, ginger.

Preferably, the plant tissue-coating medium comprises alginate and/or xanthan.

According to preferred embodiments of any one of aforementioned aspects, the plant tissue fragment regenerate into a plant with a high efficiency.

Preferably, the plant tissue fragment has a mean size of between about 0.5 mm and about 20 mm.

More preferably, the plant tissue fragment has a mean size of between about 2 mm and about 4 mm.

Even more preferably, the plant tissue fragment has a mean size of about 3 mm.

In particular preferred embodiments, the plant tissue fragment has a mean diameter size, and more preferably a mean diameter size in each direction.

In preferred embodiments of any one of the aforementioned aspects, by culturing is meant “in vitro” culture.

In any one of the aforementioned aspects, the plant fragments and preferably the plant meristematic tissue fragments, regenerate into plants or plant tissue without intervening callus or somatic embryo production.

In preferred embodiments of any one of the aforementioned aspects, the plant tissue or plant meristematic tissue is of a monocotyledonous plant or dicotyledonous plant. Preferably, the plant tissue or plant meristematic tissue is of a monocotyledonous plant.

In particularly preferred embodiments, the plant tissue or plant meristematic tissue is of a monocotyledonous plant. In preferred embodiments, the monocotyledonous plant is selected from a plant of the Poaceae family, a plant of the Poaceae family of the Musa family and a plant of the Zingiberaceae family.

Preferably, the monocotyledonous plant is of the Poaceae family which includes sugarcane and cereals such as wheat, rice, rye, oats, barley, sorghum and maize. More preferably, the monocotyledonous plant is selected from the group consisting of sugarcane, sorghum and wheat.

Other monocotyledonous plants which are contemplated include bananas, HHes, tulips, onions, asparagus, ginger, bamboo, oil palm, coconut palm, date palm and ornamental palms such as kentia and rhapis palms.

In other preferred embodiments, the monocotyledonous plant is of the Musa family and more preferably, banana.

In yet other preferred embodiments, the monocotyledonous plant is of the Zingiberaceae family and more preferably, ginger.

Also contemplated are cells, tissues, leaves, fruit, flowers, seeds and other reproductive material, material useful for vegetative propagation, F1 hybrids and all other plants and plant products derivable from said monocotyledonous plant.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF FIGURES

In order that the invention may be readily understood and put into practical effect, preferred embodiments will now be described by way of example with reference to the accompanying:

FIG. 1 Apical bud and meristem pieces after culturing and ready for tissue processing steps.

FIG. 2 Proliferating micro-shoot clusters cleaned of excess agar, leaf growth and brown tissue.

FIG. 3 Fragmented tissue produced by the plant tissue processing apparatus.

FIG. 4 Perspective view of laboratory-scale artificial plant seed production apparatus according to an embodiment of the present invention.

FIG. 5 Different stages and germination of growth of an artificial plant seed into a plantlet over 3 weeks in liquid culture.

FIG. 6 Plant regeneration response of artificial seeds of sugarcane cultivar KQ228 grown on Murashigc and Skoog (MS) medium with or with out different auxins. IBA—indole-3-butyric acid; NAA—α-napthaleneacetic acid. Error bars indicate ±s.e

FIG. 7 Plant regeneration response of artificial plant seeds of sugarcane cultivar Q208 grown on MS medium with or with out different auxins. IBA—indole-3-butyric acid; NAA—α-napthaleneacetic acid. Error bars indicate ±s.e

FIG. 8 An artificial plant seed with shoot and root development. The gel matrix is still attached to the base of the plantlet on the right (as shown by the arrow).

FIG. 9 A comparison of plant regeneration from artificial plant seeds of 4 commercial varieties. Error bars indicate ±s.e.

FIG. 10 Laboratory-scale artificial plant seed production apparatus for sugarcane artificial plant seed production (left). Close up view (right) of artificial plant seeds directly after removal from lower chamber.

FIG. 11 Effect of tissue coating matrix ratio on artificial plant seed production of sugarcane cultivar KQ228. Legend per grouping first bar=total; second bar=usable; third bar=empty. Error bars indicate ±s.e.

FIG. 12 Artificial plant seed regeneration from 3 tissue types obtained from micro-shoot clusters. Error bars indicate ±s.e.

FIG. 13 Average size (mm) of artificial plant seeds containing tissue fragment

FIG. 14 Refinement of fragment size was needed to improve the production of useful artificial plant seeds

FIG. 15 Perspective view of a plant tissue processing apparatus according to a preferred embodiment of the present invention.

FIG. 16 Plan view of a plant tissue processing apparatus showing blades and pushers according to an embodiment of the present invention.

FIG. 17 (A) Perspective view of a cutting chamber from a plant tissue processing apparatus according to an embodiment of the present invention; (B) Plan view of the cutting chamber of (A); (C) Sectional view of cutting chamber through lines indicated in FIG. 17 (B).

FIG. 18 Sectional view of the cutting chamber through lines J to J.

FIG. 19 Sectional view of the cutting chamber through lines B to B.

FIG. 20 (A) Plan view of the cutting chamber; (B) Sectional view of the cutting chamber through lines as indicated.

FIG. 21 Shoot apical and axillary buds cultured in vitro (A) develop into proliferating clusters of meristematic tissue (B). Fragments of (B) are capable of developing into shoots or plants in vitro.

FIG. 22 Proliferating meristematic tissue (A) were sliced into to 2 or 3 mm² fragments (B) capable of regenerating plants in vitro.

FIG. 23 Plant regeneration potential for different parts of proliferating meristematic tissue mass. Effect of tissue fragment size and the method of fragment production [hand-cut (HC) vs coffee mill (CM)] on sugarcane plant regeneration. Four replicates per treatment. Each replicate (flask) contained 40 artificial seeds in liquid MS medium supplemented with 4 μM BA. Cultures were maintained in shaker (120 rpm), 16 hr photoperiod and at 27° C. 1 g of meristematic tissue produced on average 49 artificial seeds.

FIG. 24 Regenerative capacity of different parts of proliferating meristematic tissue used for tissue fragment production. Four replicates per treatment. Each replicate (flask) contained 30 artificial seeds in liquid MS medium supplemented with 4 μM BA. Cultures were maintained in shaker (120 rpm), 16 hr photoperiod and at 27° C. 1 g of tissue produced on average 49 artificial seeds.

FIG. 25 Optimisation of encapsulation matrix for artificial seed production using tissue fragments from shoot tip or axillary bud-derived proliferating meristematic tissue. Ten replicates per treatment. Each replicate (flask) contained 35 artificial seeds or 1.4 g of 3 mm³ tissue fragments in liquid MS medium supplemented with 4 μM BA. Cultures were maintained in shaker (120 rpm), 16 hr photoperiod and at 27° C.

FIG. 26 Germination of artificial seeds and plantlet development over 4 weeks (A). Plantlets produced from artificial seeds growing in soil substrate (B).

FIG. 27 Germination and establishment of sugarcane artificial seeds in soil. They were grown in glasshouse. Artificial seeds were sowed either at 1 or 2 cm deep or kept uncovered with soil. Each treatment had 10 replicates. Every week plantlet germination was recorded. The artificial seeds were pre-cultured in liquid MS medium supplemented with 0.5 μM NAA for 2 weeks, on shaker 120 rpm, 16 hr photoperiod, and at 27° C. Legend: first bar=covered with 1 cm soil; second bar=uncovered with soil; third bar=covered with 2 cm soil. Error bars indicate ±s.e.

FIG. 28 Tissue fragments suspended in alginate-kelzan suspension (A). Bench-scale immobilisation apparatus for sugarcane artificial seed production (B); note the artificial seeds are formed in the lower chamber. Artificial seeds ready for germination (C).

FIG. 29 Determining the optimum tissue: encapsulation matrix ratio for production artificial seeds. Legend: first bar=beads with fragments; second bar=empty beads; third bar=distorted beads.

FIG. 30 A droplet with a tissue fragment forming from the upper chamber of the bench-scale immobilisation machine (A) artificial seeds containing tissue fragments.

FIG. 31 Tissue processing machine and the specifications (A and B); machine cut fragments 5 days (C) and 4 weeks (D) after culturing on basal nutrient medium. Regeneration of fragments occurred on basal nutrient medium.

FIG. 32 Comparison of meristematic tissue fragment production using tissue processing machine and manual hand cutting method. Manual process minimised tissue damage and hence yielded more useful tissue fragments compared to mechanical fragmentation.

FIG. 33 Plant regeneration from artificial seeds of 4 commercial varieties. Each flask contained fragments produced from 7 gm of meristematic tissue. Error bars indicate ±s.e.

FIG. 34 Auxin-induced improvement in conversion of artificial seeds into plantlets of two most commercially important Australian sugarcane varieties (Q208 and KQ228)

FIG. 35 Comparison of plant regeneration efficiency of ginger meristematic fragments and artificial seeds after growing for 6 weeks in liquid culture

FIG. 36 Comparison of plant regeneration efficiency of ginger meristematic fragments and artificial seeds after growing for 3 weeks in liquid culture

FIG. 37 Flow chart of key steps involved in sugarcane artificial seed production technology Legend 1. Shoot top, the source of shoot apical and axillary meristems; 2. Proliferating meristematic tissue obtained from shoot tip and/or axillary bud; 3. Tissue Processing Machine for fragmenting meristematic tissue. 4. Fragmented meristematic tissue 5.

Fragmented meristematic tissue in alginate-kelzan suspension; 6. Production artificial seeds in the immobilization apparatus; 7. Artificial seeds. 8 Germinating artificial seeds 9. Plantlets produced from artificial seeds planted in the field

### DETAILED DESCRIPTION OF THE INVENTION

The present invention is predicated, at least in part, on the development of methods and systems for preparation of plant tissue fragments that are able to regenerate into a plant or plant tissue that overcomes high production costs of other micropropagation technique yet is highly efficient. In other broad aspects, the present invention is predicated, at least in part, on the development of an artificial plant seed system that utilises small fragments of plants and in certain embodiments, micro-shoot clusters, derived from proliferating sugarcane axillary buds and/or shoot apex in vitro, to produce plantlets, although it will be appreciated that the invention can be extended beyond sugarcane to monocots and dicots. In particular broad aspects, the invention provide methods and systems for preparation of plant meristematic tissue fragments. In particular embodiments, the methods or systems of the present invention produce a plant meristematic tissue fragment or plant tissue fragment that is able to regenerate into a plant or plant tissue. In particularly preferred embodiments, plants or plant tissue may be regenerated directly from the fragments produced by the invention without intervening callus or somatic embryo production. A particular advantage provided by the fragments of the invention is successful production of plants in high frequency (80-90%) directly from small fragments.

Plant tissue culture has been used extensively in plant propagation, transformation, mutagenesis, breeding and virus elimination. Such tissue culture systems me generally referred to as “micropropagation” systems, wherein plant tissue explants are cultured in vitro in a suitable solid or liquid medium, from which mature plants are regenerated. In particular embodiments, “micropropagation” relates to conventional micropropagation technology or alternatively, artificial plant seed technology. As will be appreciated by a person of skill in the art, conventional micropropagation technology includes micropropagation techniques that do not include production of an artificial plant used but relates to propagation and regeneration of plants and plant tissues from an in vitro cultured plant, plant tissue and/or parts thereof.

By “artificial plant seed” is meant a plant seed which does not occur in nature but rather is a propagule functionally similar to a plant seed that has been produced by some level of human intervention using micropropagation techniques. The “artificial plant seed” is able to regenerate into a plant and may undergo germination. The terms “artificial plant seed” and “artificial seed” may be used interchangeably herein.

In particular broad aspects, the invention resides in methods of preparing plant meristematic tissue fragments for use in plant micropropagation by (i) inhibiting apical dominance of a plant meristematic tissue; and (ii) processing the plant meristematic tissue resulting from step (i) to prepare a plant meristematic tissue fragment that is suitable for use in plant micropropagation as exemplified in the Examples section and in particular, Examples 1, 3 and 7-10. The plant meristematic tissue fragments prepared by these methods are suitable for use in conventional plant micropropagation technology or artificial seed technology.

Broadly, step (i) that includes inhibition of apical dominance results in the production of genetically uniform propagules (or otherwise known as “true-to-type propagules”) from a plant meristematic tissue and preferably, large quantities of organogenically competent plant meristematic tissue for use in step (ii). In preferred embodiments, step (i) includes in vitro culture and proliferation of plant meristematic tissue without differentiation into shoots or plantlets. The ability to produce and maintain meristematic tissue capable of regenerating into shoots or plantlets for extended periods under defined culture conditions is achieved by inhibiting apical dominance and thus allowing axillaries to proliferate.

In preferred embodiments, the plant meristematic tissue is derived from shoot apical meristem tissue or alternatively, axillary meristem tissue. It will be appreciated by a person of skill in the art that apical bud meristem tissue is derived from shoot apex whilst axillary meristems is derived from axillary buds from the primary or axillary shoot apical meristem.

“Apical dominance” is a term used in the art whereby vertical growth supercedes lateral growth in a plant. Apical dominance is controlled by plant hormones calledauxins.

The present invention contemplates inhibition of apical dominance. In the context of the present invention, by “inhibit”, “inhibition”, “inhibited”, “inhibitory” or “inhibitor” is meant any treatment which at least partly interferes with, prevents, abrogates, suppresses, reduces, decreases, disrupts, blocks or hinders dominant vertical growth of a plant or plant tissue resulting from the plant apex or plant tissue apex and includes full inhibition of apical dominance. By way of example, “inhibition” can refer to a decrease of about 10%, 20%, 30%, 40%, 50%, 60%, 70%, 80%, 90% or 100% in apical dominance.

Apical dominance may be inhibited by any one or a plurality of means as are known in the art. Physical treatment includes mechanically abrogating growth of the apical bud tissue by severing or cutting the apical bud, although without limitation thereto. Accordingly, removal of dominance of the primary shoot may occur by excising the apical bud. In preferred embodiments, apical dominance is inhibited by longitudinal slicing of the plant meristematic tissue.

The invention also contemplates chemical inhibition of apical dominance by hormone treatment or use of other small organic molecules with a desired biological activity and half-life.

The invention further contemplates biochemical techniques for apical dominance inhibition inclusive of molecular and genetic techniques. Non-limiting examples of molecular inhibition of apical dominance include use of peptides, proteins such as antibodies. Genetic techniques include use of nucleic acid or gene based technologies which include use of ribozymes, gene silencing molecules such as miRNA, siRNA and the like.

In certain preferred embodiments, the plant meristematic tissue is cultured or propagated prior to inhibition of apical dominance. The period of culture is as required and can be up to about 1 week, about 2 weeks and about 3 weeks although without limitation thereto. In particularly preferred embodiments, the plant meristematic tissue is cultured in vitro. In particularly preferred forms of these embodiments, the plant meristematic tissue is derived from shoot apical meristem tissue although use of axillary meristem is also contemplated.

In other certain embodiments, apical dominance of the plant meristematic tissue is inhibited prior to culture. According to these embodiments, the plant meristematic tissue is cultured whilst maintaining inhibition of apical dominance.

In preferred embodiments, the plant meristematic tissue is cultured under conditions of inhibition of apical dominance until re-emergence of apical dominance ie. until first shoot formation. The period for culture is as required to generate desired quantities of plant meristematic tissue and preferably, up to about 1 week, about 2 weeks, about 3 weeks, about 4 weeks, about 6 weeks, about 8 weeks, about 3 months, about 4 months, about 5 months, about 6 months, about 7 months, about 8 months, about 9 months, about 10 months, about II months and about 12 months or more as long as the tissue remains meristematic.

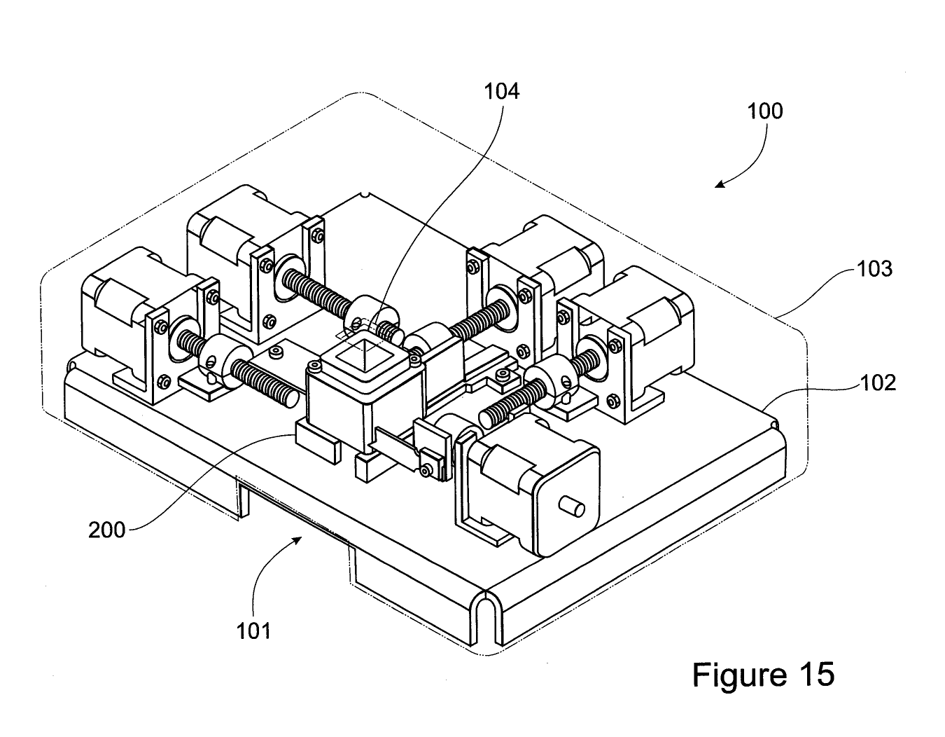

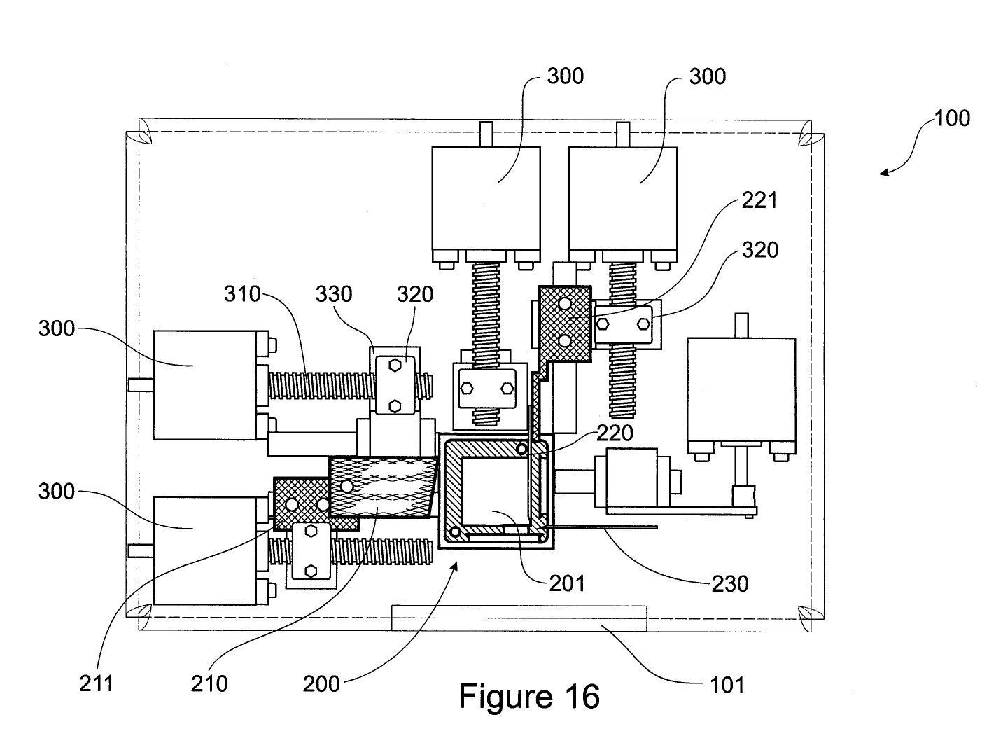

In particular embodiments, the step of fragmenting a plant meristematic tissue of step (ii) is by way of severing, slicing or otherwise cutting. The step of fragmentation may be performed manually with a conventional knife or may be automated or semi-automated (such as using a milling machine such as a coffee mill) or undertaken by an automated device. In preferred embodiments, step (ii) is performed by the plant tissue processing apparatus as depicted in FIGS. 15 and 16.

In particular embodiments, the plant meristematic tissue is not derived from fern.

In preferred embodiments, the dead tissue is removed prior to the fragmenting step.

### Automated Tissue Processing Machine

In other broad aspects, machines have been developed to automate the labour-intensive steps of this process. This includes an apparatus for fragmenting proliferating masses of micro-shoots and an automated system for encapsulating those fragments. The artificial plant seeds developed using micro-shoots are capable of growing into normal, well-developed plantlets two (2) weeks after placing into a liquid culture system. The present invention is particularly amenable in systems in which embryogenesis cannot be used for micropropagation and need to rely on other forms of morphogenesis. Therefore a non-exclusive underlying motivation of the present invention is to produce clonal material using a technology that leads to proliferation of meristems (the so called plant stem cells) and adapting that to artificial plant seed production technology. Accordingly, the inventors have conceived and developed an apparatus and system that produce sterile, morphogenically-competent target tissues for rapid production of material for artificial plant seed production, and regeneration of plants. This has considerable commercial value.

A particular advantage, although without limitation thereto, of the invention is at least partial automation, semi-automated or fully automated system of shoot meristem-based plant micropropagation which has the ability to produce clonal (true-to-type) propagules more than any other in vitro propagation technologies (callus culture, cell culture, protoplast culture, direct organogenesis, somatic embryogenesis, etc).

Therefore according to broad aspects of the present invention, the invention is broadly directed to a plant tissue processing apparatus for generating plant tissue fragments suitable for use in plant micropropagation. In particularly preferred embodiments, the plant tissue fragments produced therefrom are suitable for use in an artificial plant seed, wherein the artificial plant seeds regenerate plants with a high efficiency. In a particular form, the plant tissue processing apparatus is a plant tissue cutting apparatus.

The invention is also broadly directed to methods of plant micropropagation and/or artificial seed production which utilises the plant tissue processing apparatus.

FIGS. 15 and 16 shows a plant tissue processing apparatus 100 according to an embodiment of the present invention. The plant tissue processing apparatus 100 comprises a cutting chamber 200 and a plurality of driving motors 300. As will be appreciated by the skilled addressee, the power source for the operation is taken direct from single phase electrical supply. The power is stepped down by a transformer before being supplied to the driving motors 300. The driving motors 300 are connected to square threaded shafts 310 which in turn have a brass nut 320 attached. The brass nut 320 is fixed to a tool holder 330 and moves along the length of the shaft 310, which in turn drives the tool holder 330 in and out of the cutting chamber 200. The tool holder 330 move on linear bearing assemblies. As will be described in more detail hereinafter, a blade is driven by a bell crank arrangement and moves on linear bearing assemblies, whilst another is attached to a lead screw nut and moves back and forth on linear bearings. Cutting of plant tissue take place within the cutting chamber 200 and collection of the cut plant tissue fragments takes place in the collection tray 101.

it will be appreciated that in a preferred embodiment, a programmable logic controller controls the operating sequence of the plant tissue processing apparatus 100. As can be seen in FIG. 15, the apparatus mechanism are preferably mounted on a machined aluminium base 102 and covered by a clear Perspex cover 103. The purpose of the cover is two fold: (1) to provide a safety barrier between the machine whilst in operation and the operator. The transparency of the cover allows for monitoring of operation without exposure of personnel to the mechanism of the apparatus; (2) the cover enables the control of sterility of the operating environment during operation. The plant tissue sample to be cut is introduced through the cover in a specially designed feeder tube 104. Pressure is applied to the raw material by the introduction of a light weight on top of the material in the feeder tube. During and on completion of the cutting operation samples can be collected from an opening 101 situated under the apparatus without removal of the cover.

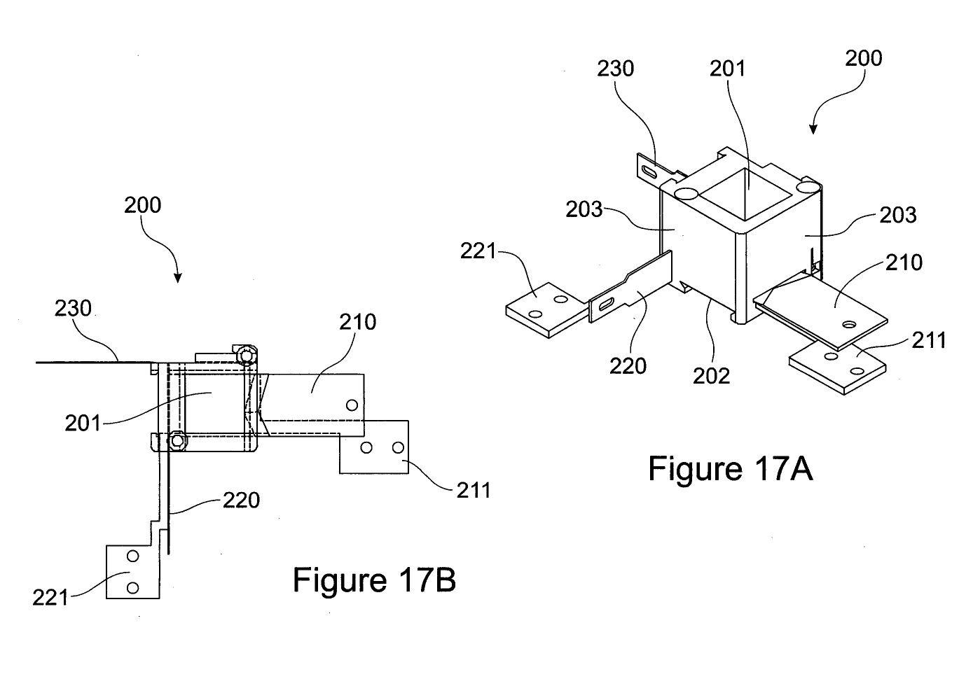

FIGS. 17A and 17B shows a more detailed view of the cutting chamber 200. The cutting chamber 200 comprises an aperture 201 formed through vertical side walls 203 and a floor 202 into which the plant tissue is loaded for subsequent cutting. The cutting chamber 200 further comprises a first blade 210, a second blade 220 and a third blade 230. Associated with the first and the second blades are a first pusher 211 and a second pusher 221 respectively.

The first blade 210 slices a plant tissue directly from loading. The first pusher 211 pushes the material at low torque full length into the aperture 201 of cutting chamber 200. The second blade 220 cuts the plant tissue cut by the first blade 210 to size in one dimension. The second pusher 221 pushes the plant tissue further into the aperture 201 of cutting chamber 200. The third blade 230 cuts the plant tissue to its final desired fragment size. In this way, a plant material or plant meristematic tissue of the present invention is severed in an ordered sequence by at least two blades along at least two different planes. By “severed in an ordered sequence” is meant to sever, fragment, slice or otherwise cut in an ordered manner and thus not in a random manner. In particular preferred embodiments, “severed in an ordered sequence” is severing a plant tissue or plant meristematic tissue sequentially. Although it will be appreciated that in other certain embodiments, the plant tissue or plant meristematic tissue is severed non-sequentially by at least two blades yet in an ordered sequence. The plant tissue fragments are subsequently collected in a tray under the plant tissue processing apparatus 100.

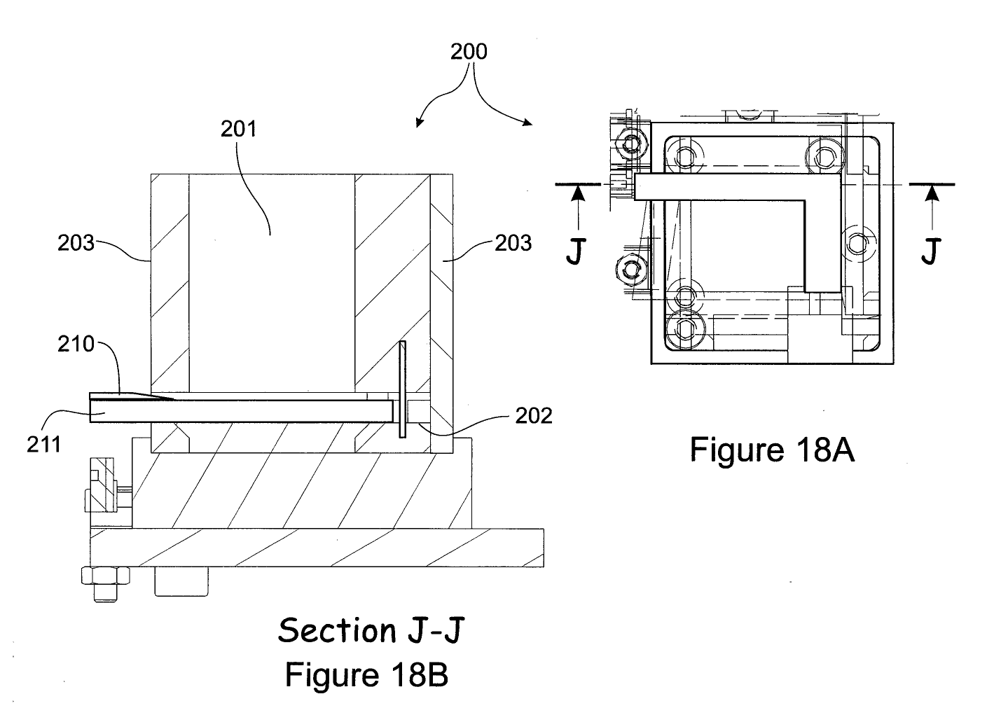

FIG. 18 shows a sectional view through lines J to J of the cutting chamber 200. In operation, the first blade 210 enters the aperture 201 through a plane that is about parallel to the floor 202 of cutting chamber 200 and makes a full cut of the plant tissue. That is, the first cut of the plant tissue with the first blade 210 may generate a slab of the plant tissue. The slab of the plant tissue is pushed by the first pusher 211 further into the cutting chamber in preparation for the second cut.

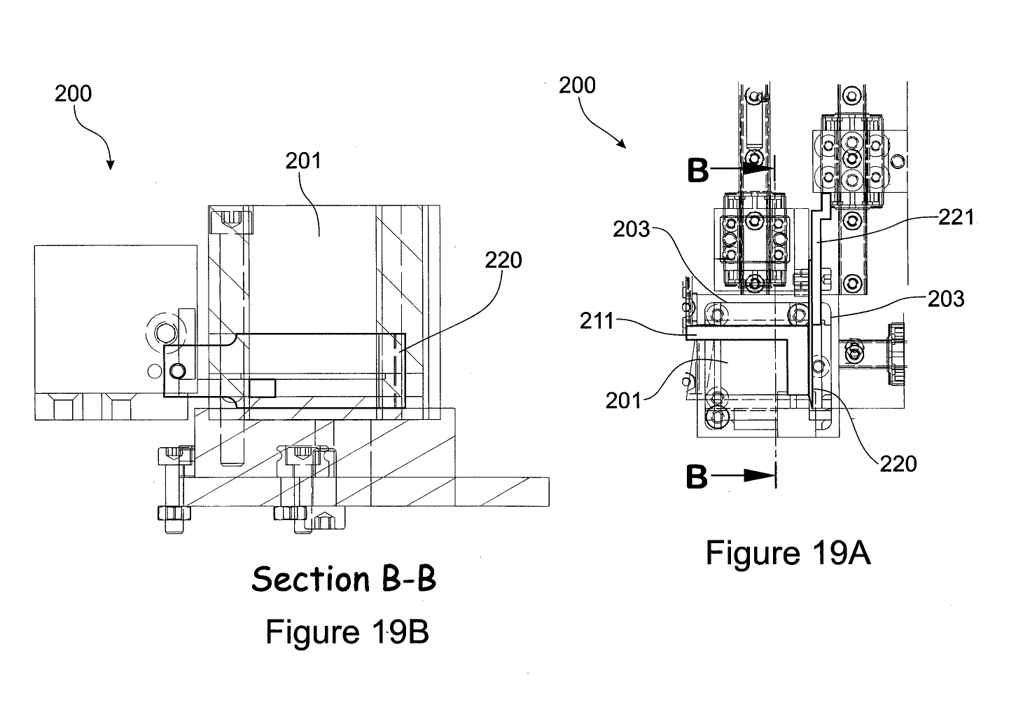

FIG. 19 shows a sectional view through lines B to B of the cutting chamber 200. The second blade 220 enters the cutting chamber 200 at a plane that is about perpendicular to the vertical side walls 203 and thus essentially cuts the slab of the plant tissue generated by the first cut into a strip. The second pusher 221 subsequently pushes the strip of plant tissue before the third blade 230.

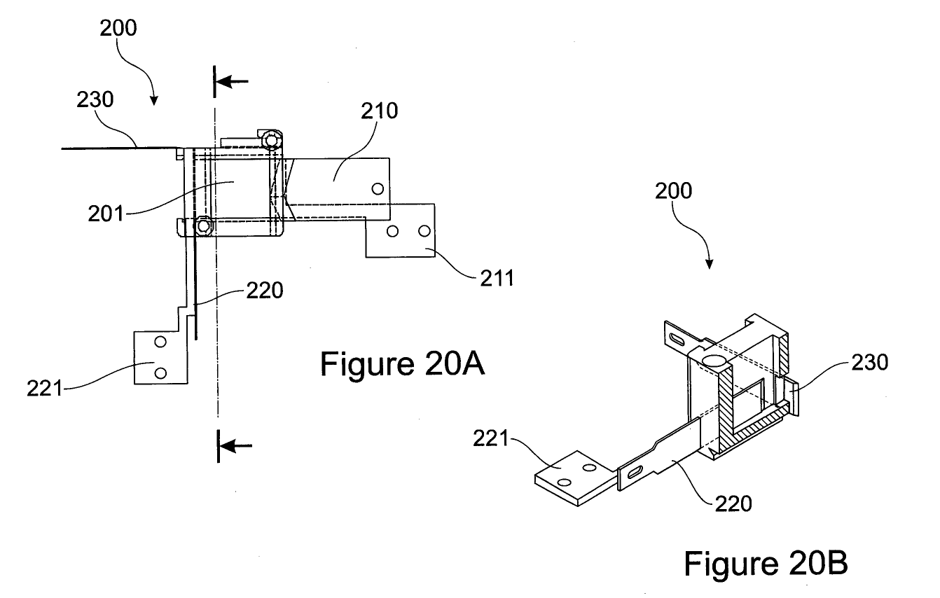

FIG. 20 shows the third blade 230 with respect to the cutting chamber 200. The third blade 230 is positioned with respect to the cutting chamber 200 at about perpendicular to the vertical side walls 203. The third blade 230 rapidly cuts the strip which is being pushed by the second pusher 221 into fragments of a desired size and shape. For example, the fragments may be a cube, although without limitation thereto. A skilled addressee will appreciate that the fragments produced will have the size and/or integrity such that plant tissue fragments that do not require a developmental stage on culture media prior to coating of the plant tissue fragment. Moreover, approximately equal sized fragments are produced under aseptic conditions with minimal user handling. Further advantages is that the apparatus is conducive to mass plant production and there is little or no damage to the tissue which then does not reduce plant regeneration rates. The fragments generated by the third blade 230 are subsequently collected for further processing.

The present invention as it applies to the plant tissue processing apparatus 100 is applicable to a number of different plant tissues inclusive of leaf spindle or whorl, leaf blade, axillary buds, stems, shoot apex, leaf sheath, internode, petioles, flower stalks, embryo, root or inflorescence. Suitably, a relevant biological property of the plant tissue used in the present invention is that they contain actively dividing cells having growth and differentiation potential. Preferably, the plant tissue is axillary bud and/or shoot apex. In preferred embodiments, the shoot apex is apical bud tissue and/or apical meristem tissue.

it will be appreciated that the plant tissue fragments generated by the plant tissue processing apparatus 100 or otherwise generated by step (ii) as hereinbefore described should have a mean size, and preferably a mean diameter size, which is conducive to production of an artificial plant seed or in the case of conventional plant microproagation, conducive to regenerate into a plant or plant tissue according to the methods of the present invention. In preferred embodiments, the mean size is about 0.5 mm, about 1 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10.5 mm, 11 mm, 11.5 mm 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm, 18.5 mm, 19.0 mm, 19.5 mm and 20.0 mm. In particular embodiments, the preferred mean size is about 3 mm.

In other broad aspects, the invention provides methods of producing an artificial plant seed which does not require a development stage on tissue culture media after fragmentation and prior to encapsulation of the tissue fragment into a plant tissue-coating medium.

In certain preferred embodiments, the methods of producing artificial plant seeds of the present invention that include use of the plant tissue processing apparatus 100 further includes the steps of culturing a plant tissue prior to fragmentation using the plant tissue processing apparatus 100. Suitably, the plant tissue derived from a plant is cultured in vitro with growth media, preferably with its cut side down, for a sufficient period to allow the plant tissue to reach an explant size that is able to be subsequently processed. A preferred culture period is 4 weeks however it will be appreciated that the culture time may vary depending on a number of factors such as plant tissue type and may be lengthened or shortened as required.

Prior to processing in the plant tissue processing apparatus, the cultured explant is cleaned by removal of leaf tissue and any dead tissue, and if required, excess agar. It will further be appreciated that the in vitro culture may be performed on solid or liquid medium.

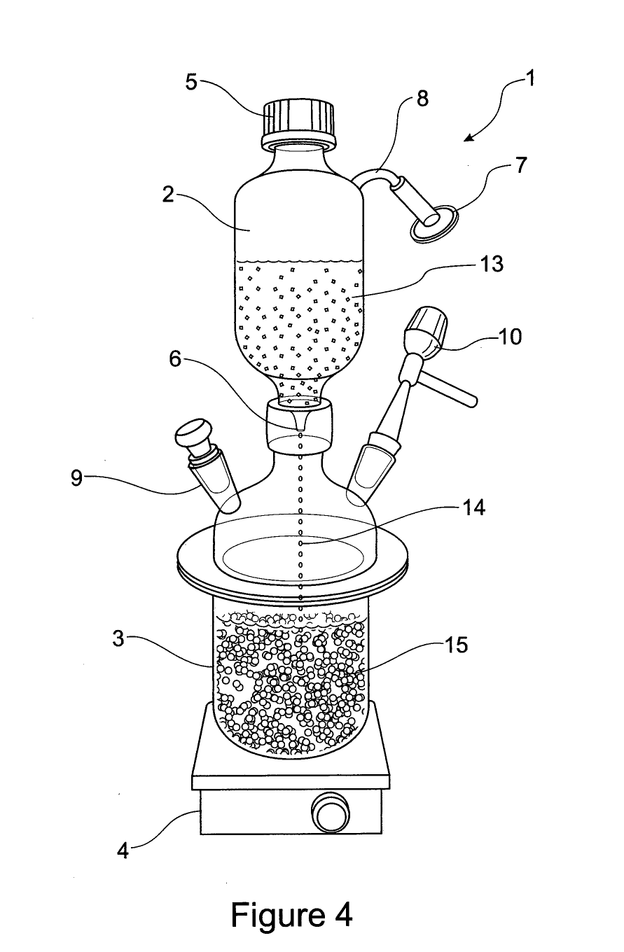

FIG. 4 depicts an artificial plant seed production apparatus 1 according to an embodiment of the present invention. The artificial plant seed production apparatus 1 comprises a first chamber 2, a second chamber 3 and a stirrer unit 4. The first chamber 2 comprises an entry point 5 and an orifice 6 located at opposite ends of the first chamber 2. A filter 7 and a filter joint 8 are located at a side the first chamber 2. The second chamber 3 comprises a glass seal 9 projecting from an upper point and a stop valve 10 located opposite. The first chamber 2 and the second chamber 3 are associated with each other such that the orifice 6 discharges material in the second chamber 3.

In operation, plant tissue fragments are mixed with a plant tissue-coating medium outside the fist chamber 2. The mixture 13 is poured through the entry point 5 and the lid of the first chamber is placed on to thus create a seal and an internal vacuum. The stirrer 4 is switched on to create a vortex of about 2 cm in height of the seed coating-setting solution. The stop-valve 10 is then opened slowly to allow sufficient flow of the plant tissue fragment mixture 13 through the orifice 6. Single droplets 14 of the mixture drop descend from the first chamber 2 into the second chamber 3. When the droplets 14 from the first chamber 2 mix with the seed-coat setting solution in the second chamber 3, the droplets set into an artificial plant seed containing the plant tissue fragment 15. The artificial plant seeds 15 remain stirring in the second chamber 3 for sufficient time to allow the coating medium to fully harden. The artificial plant seeds are subsequently decanted off and rinsed, preferably in sterile deionised water, to thereby produce an artificial plant seed. The artificial plant seed can be sold without plantlet propagation or alternatively, the artificial plant seed can be germinated and cultured to produce a plantlet which can subsequently be sold to an end-user.

it will be appreciated that an advantage of the artificial plant seed production apparatus 1 is that a number of artificial plant seeds can be generated in a short period. Moreover, the need for operator input is minimised.

it is appreciated that the plant tissue-coating medium can comprise any polymer, solute, carbohydrate, guar gum, carrageenan (and combinations thereof) that are suitable for coating or encapsulation of a plant tissue to produce an artificial plant seed. Preferably, the plant tissue-coating medium comprises sodium alginate and xanthan. In particularly preferred embodiments, the concentration of sodium alginate is 3-4% w/v whilst the concentration of xanthan is 1-1.5% w/v, this concentration being the concentration of the solution added to the plant tissue-coating medium. In particularly preferred embodiments, the concentration of sodium alginate is about 3% w/v whilst the concentration of xanthan is about 1% w/v. It will be appreciated that the concentration of agents used in the plant tissue-coating medium will vary depending an the agent that is used and the ratio of plant tissue to plant tissue-coating medium. In particularly preferred embodiments, the plant-tissue coating medium will be at a concentration that will produce at least 5%, 10%, 20%, 30%, 40%, 50%, 60%, 70%, 80%, 90%, 95% or 100% efficiency of regeneration or germination into plantlets.

it will be appreciated that sodium alginate is commercially available as Manugel GMB® whilst xanthan is available as Kelzan®, as are other potentially useful plant tissue-coating formulations.

In preferred embodiments, the seed cost-setting solution is CaCl₂ at a particularly preferred concentration of 0.06M. However the skilled addressee will appreciate that any seed coat-setting solution may be used and to a certain extent the choice of seed coat-setting solution is dependent upon what is used for the plant tissue-coating medium. It is appreciated that the plant tissue-coating medium can comprise chemicals such as ferric chloride, cobaltous chloride, calcium nitrate and calcium hydroxide.

In those embodiments which contemplate culturing of plant tissue or plant part, the culture medium may include Murashige and Skoog nutrient formulation (Murashige and Skoog, 1962, Physiologia Plantarum 15; 473) or Gamborg's medium (Gamborg et al, 1968, Exp. Cell. Res. 50: 151). Preferably, the medium comprises Murashige and Skoog nutrient formulation. It will be appreciated that the abovementioned media me commercially available, as are other potentially useful media.

it will be appreciated that the culture media may contain further supplements required for growth of the explant such as, but not limited to, sugars, hormones (eg. auxins and cytokinins), citric acid and ascorbic acid. Reference is made to International Publication No. WO 01/82684 (incorporated by reference) which provides non-limiting examples of suitable growth media and supplements which can be applied to the present invention.

it is also preferred to have an ideal ratio of tissue to setting solution so that the immobilisatlon apparatus operates optimally. In particularly preferred embodiments that relate to alginate, the ratio of tissue to solution may be between 50 g and 100 g of tissue/L and most preferably, 70 g tissue/L.

Although the present invention is preferentially exemplified using sugarcane, ginger and banana, it will be appreciated that the invention can be applied to any plant inclusive of monocotyledonous plants and dicotyledonous plants. In certain preferred embodiments, the invention is particularly directed to members of the Poaceae family inclusive of sugarcane, cereals, wheat, sorghum and maize, and other plants such as pineapple, orchids, oil palm, date palm and Miscanthus sp.

In other broad aspects, the invention relates to a system for plant micropropagation in which an apparatus fragments a plant tissue, and preferably a plant meristematic tissue that has undergone inhibition of apical dominance, followed by coating of the plant fragment. In preferred embodiments, the system includes a plant tissue processing apparatus to produce the fragments. The system may also include an artificial seed production apparatus to coat the plant fragment in plant tissue-coating medium.

Preferably, the system is an integrated system.

Preferably, the system includes the plant tissue processing apparatus 100 and/or the artificial plant seed production apparatus 1.

Preferably, the system includes one or more elements selected from features 3 to 6 of FIG. 37. The system can be semi-automated or fully-automated.

So that the invention may be readily understood and put into practical effect, the following non-limiting Examples are provided.

### EXAMPLES

### Introduction

Sugarcane is a major crop of Australia, generating export revenue of around $941 million annually (Australian Bureau of Agricultural and Resource Economics 2009). Commercial sugarcane is propagated vegetatively by stem cuttings called billets. In Australia, about 20% of the crop (about 80,000 ha) is replanted every year (Australian Sugar year Rook 2008). It is estimated that about 880 million plantlets are required annually for replanting. Production of disease-free plantlets at this scale is highly laborious and uses 6-10 t/ha of millable stalks (worth nearly $17 million/year for the whole industry) that otherwise could be used for sugar production. In an effort to deliver higher productivity, a more efficient, automated micropropagation method for large-scale production of planting material was sought. In Brazil, Syngenta has developed a method of producing sugarcane nodal stem segments of less than four centimetre in length—Plene. These are treated with proprietary crop protection and seed care products to maximize early plant development and crop establishment it is claimed that Plene will allow sugarcane growers to replant their fields more frequently, eliminating the typical yield degradation of the crop and thereby leading to a yield gain of up to 15%. It would also enable growers to use lighter planting equipment which saves on fuel costs. However, planting machinery is still under development for this process.

Rapid and efficient tissue culture based systems for commercial sugarcane are not new. Lakshmanan et al. (2001) developed a rapid and efficient in vitro regeneration method using a transverse thin cell layer culture system, called Smartett™, for production of large quantities of cultivars for commercial planting in Australia. Sugarcane industries in Brazil, Cuba, India, and USA already use micropropagation for producing planting material for commercial use. However, the cost of seedlings produced is much higher than the conventional billet-derived material, limiting its adoption by the industry.

In an effort to reduce labour, much work on the automation of micropropagation of somatic embryo-derived plant products has been done (Guiderdoni et al., 1995). Although developed originally as an alternative regeneration system to meristem culture, somatic embryogenesis has achieved prominence as an integral part of the genetic transformation system (Bower and Birch, 1992). Somatic embryogenesis has been reported from a large number of commercial sugarcane clones (Guiderdoni et al., 1995; Manickavasagam and Ganapathi, 1998), and can be obtained directly (Manickavasagam and Ganapathi, 1998), or indirectly (Guiderdoni and Demarly, 1988), from the leaf tissue. Embryogenic callus can be maintained for several months without losing its embryogenic potential to any significant level (Fitch and Moore, 1993).

Genetic variability has been frequently reported in tissue-cultured sugarcane (Heinz and Mee, 1971; Lourens and Martin, 1987; Burner and Grisham, 1995; Taylor et al., 1995; Hoy et al., 2003). Studies were conducted to assess the extent of variability arising from in vitro regeneration and its transmission into successive generations via vegetative propagation (Lourens and Martin, 1987; Burner and Grisham, 1995). These investigations demonstrated that substantial somaclonal variability occurred in in vitro-derived propagules, irrespective of the method of regeneration. However, extensive field experiments have shown that the phenotypic variations in tissue-cultured sugarcane were frequently temporary as the majority of variants reverted to the original parental phenotype in the ratoon-crops (Lourens and Martin (1987), Burner and Grisham (1995), and Irvine et al. (1991)).

Adventitious regeneration for commercial sugarcane micropropagation has been investigated as well. NovaCane® is a micropopagation process whereby sugarcane plants are multiplied in vitro, hardened off, field-planted and then propagated vegetatively. This approach can contribute to the production of certified disease-free material at improved multiplication rates. This in-vitro propagation protocol, NovaCane®, successfully, produces an abundant source of pathogen-free plants that can be efficiently hardened off. The third and final phase of the propagation procedure is to assess clonal fidelity and plant performance in the field.

Another approach, similar to NovaCane® Is to produce planting material by integrating RITA° temporary immersion systems (TIS), a semi-automated micropropagation with SmatSett™ technology (Mordocco et al., 2005). TIS has been successfully used to propagate many crops including sugarcane (Aitken-Christie and Jones 1987; Lorenzo et al. 1998; Escalano et al. 1999; Etienne and Berthouly 2002; McAlister et al. 2005). Most of the reported TIS studies have used shoot tip, axillary bud, callus, or organs such as nodules, roots, and microtubers as the explant material (Etienne and Berthouly 2002). The sugarcane TIS systems reported so far use shoot-tip-derived cultures (Lorenzo et at. 2001; Rodriguez et al. 2003). This approach while successful and provides true-to-type clones, does not allow for sufficient scale-up and commercial use.

Presently, the inventors describe using fragmented micro-shoots clusters and an alginate encapsulation matrix to develop a sugarcane artificial plant seed production system with high plant regeneration efficiency. The axillary buds and/or shoot apex tissue is cultured for 4 weeks on semi-solid MS medium containing a cytokinin to produce proliferating masses of micro-shoots. These clusters are cleaned of extraneous leaf material and shoed to 3 mm tissue fragments and immobilised. Nearly 80% of the immobilised micro-shoots produced plantlets when maintained in an optimised MS (Murashige and Skoog) liquid medium. In addition, machines required to produce the fragment tissue and to encapsulate it into artificial plant seeds have been developed. When used in association with the protocols for adventitiously formed meristem-tissue and the artificial plant seed protocols developed, a whole system approach to produce sugarcane plantlets for commercial-scale propagation and release has been achieved.

### Example 1

### General Materials and Methods

### 1.1 Plan Materials

Young bolting sugarcane “stalk” tissue were harvested from below the apical meristem. The varieties KQ228, Q190, Q208 and Q232 were used throughout the experiments.

### 1.2 Preparation of Shoots Tops

Shoot top of 3- to 8-month-old healthy, field-grown sugarcane plants is an excellent source of explant for plant regeneration. The quality of plant material (shoot tops) plays a significant role in determining the frequency of regeneration. Shoot tops collected from stressed plants (water stress, pathogen infection, old canes, etc) do not respond well in culture. Also, avoid collecting shoot tops during rainy season to minimise contamination of culture.

### 1.3 Preparation of Axillary Buds and Apical Meristem for Tissue Propagation

Under aseptic conditions, axillary buds and apical meristem pieces were sliced from cane tops.

### 1.4 Media and Culture Conditions

Murashige & Skoog (MS) (Murashige and Skoog 1962) nutrient formulation supplemented with 30 gL⁻¹ sucrose. To form a solid medium the media was supplemented with Davis J3 grade agar (8 gL⁻¹). The basal medium was enriched with a cytokinin filter-sterilised 4 μM 6-benzylaminopurine (BA) for preparation of the axillary buds and meristem for tissue propagation. The pH of all media was adjusted to 5.7±0.1 prior to autoclaving at 121° C. and 101 kPa for 20 min. Liquid cultures were agitated continuously on a gyratory shaker at 120 rpm. All cultures were sealed with a single layer of 3M Micropore™ tape and incubated at 26° C.±2° C. with a 16 h photoperiod provided by cool white fluorescent tubes, with a photon flux density of 30 μmol m⁻² s⁻¹ at the culture level. Cultures were transferred to fresh medium once per week, or more frequently if medium or tissue turned brown due to phenolic exudation.

### 1.5 Tissue Processing

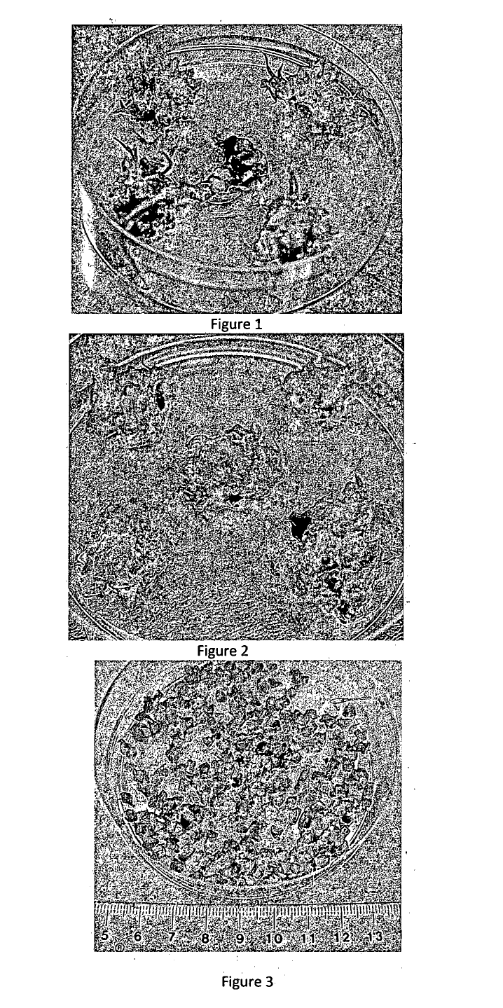

Micro-shoot clusters were removed from media plates and placed onto sterile petri dishes (FIG. 1). The tissue was cleaned of excess agar and leaf growth and brown tissue was removed by using a sterile flamed scalpel and forceps (FIG. 2). Tissue was then placed into the plant tissue processing apparatus. Tissue pieces are out into ≦3 mm shapes (FIG. 3). The tissue fragments are collected aseptically.

### 1.6 Preparation for Encapsulation of Micro-Shoot Fragments

One day prior to use, 200 mL of 3% w/v sodium alginate (manugel GMB)+1.0% w/v xanthan (Kelzan) was sterilised, cooled and placed at 4° C. overnight.

### 1.7 Assembly of the Laboratory-Sale Artificial Plant Seed Production Apparatus

The laboratory-scale artificial plant seed production apparatus was assembled in the laminar flow hood. A sterile magnetic stirrer was placed in chamber 3 with 500 mL of cold sterile 0.06 M CaCl₂ solution. This was placed onto a stirrer unit. The top of the lower chamber was greased lightly using silicon grease, and chamber 2 was placed on top. The clamp was then securely tightened onto both pieces. The glass seal and the stop-valve were also greased lightly and placed onto the smaller openings on the middle chamber. The stop-valve was closed off. Chamber 1 was greased lightly at the lower connecting joint and then placed inside chamber 2. A sterile 0.2 μm filter was placed onto the tubing attached to the filter joint.

### 1.8 Encapsulation of Micro-Shoot Fragment

Fourteen grams of fragmented 4 week-old micro-shoots was suspended in 50 mL of sterile, cold, 3% w/v sodium alginate+1% w/v xanthan. The mixture was stirred to separate the fragments and then combined with the remaining 150 mL of alginate/xanthan mixture. The mixture was poured into chamber 1 of the encapsulation apparatus and the lid placed back on and sealed. There is some spillage into chamber 2 until an internal vacuum is created.

The stirrer was switched on (medium speed) to create a vortex of approx. 2 cm in height. The stop-valve was then opened slowly to allow sufficient flow of the tissue fragment mixture through the orifice. Single droplets of mixture drop from chamber 1 into chamber 2. The stop-valve may need to be released further as the solution continues through. When chamber 1 was empty, the artificial seeds within the CaCl₂ solution were continually stirred for 30 minutes to harden. The apparatus is pulled apart and the calcium chloride decanted off from the artificial seeds. The artificial plant seeds were then washed twice with sterile DI water (500 mL) and left in the DI water until the empty and misshapen ones were removed and sorted.

### 1.9 Growth of Artificial Plant Seeds in Liquid Culture

Thirty-six artificial plant seeds (approx 15 mL) were placed into a sterile 250 mL Erlenmeyer flask with 85 mL of sterile MS liquid with 30 gL⁻¹ sucrose. Flasks were placed on the gyratory shaker trays at 120 rpm, 27° C.±1° C., and a 16 h photoperiod provided by cool white fluorescent tubes for 2-3 weeks. The media was decanted off and replaced every 3-4 days.

### Example 2

### Influence of Hormone Addition on KQ228 Plant Regeneration

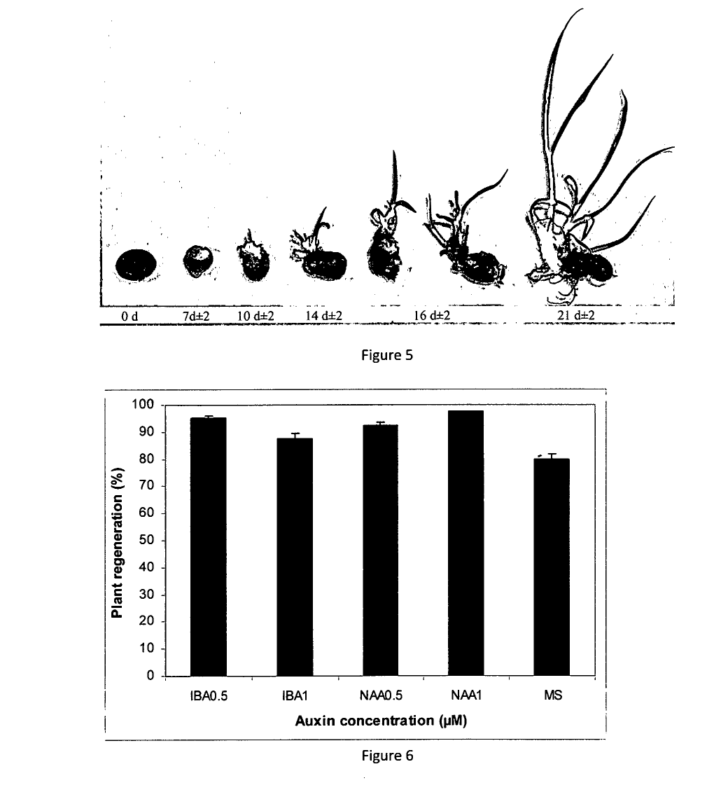

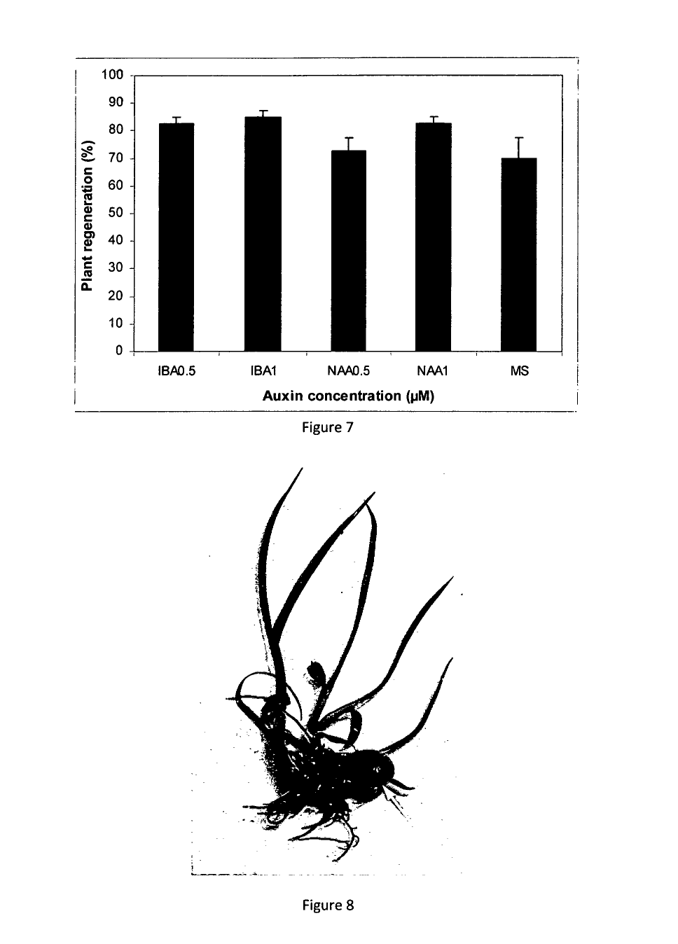

A simple liquid medium containing MS salts proved to be sufficient for germination and plantlet growth of artificial plant seeds of KQ228. In this medium the artificial plant seeds germinated and produced normal plantlets within 3 weeks (FIG. 5). The seeds change from a transparent gel bead to a brown-black colour within the first few days of being placed into liquid culture. Within 7-10 days there is shoot production from the seeds and within two weeks the shoots are elongating and root production outside of the artificial plant seed begins. Within 3 weeks the plantlet has fully developed, with both extensive shoot and root production, outside of the gel. Variations in the rate of plantlet emergence are to be expected with different genotypes (FIG. 6 and FIG. 7). Typical growth conditions for liquid culture are 120 rpm and 27° C. with a 16 hour photoperiod.

In general, plantlets produced with growth regulators were stunted if hormone levels were 1 μM compared to those obtained from growth regulator-tee MS medium. As such, MS is typically used for the liquid culture medium. Regeneration of artificial plant seeds into plantlets at a rate of 70-90% is achievable. Addition of a hormone shows increased regeneration of artificial plant seeds for both Q208 and KQ228. The artificial plaint seed system requires a 2-week period in liquid culture to germinate the seed, establish roots and shoots and grow into a plantlet (FIG. 8). This growth period occurs in flask culture or bioreactors.

### Example 3

### Adaption of Artificial Plant Seed Protocol to Different Varieties KQ228, Q232, Q190 and Q208; and the Effect on Regeneration

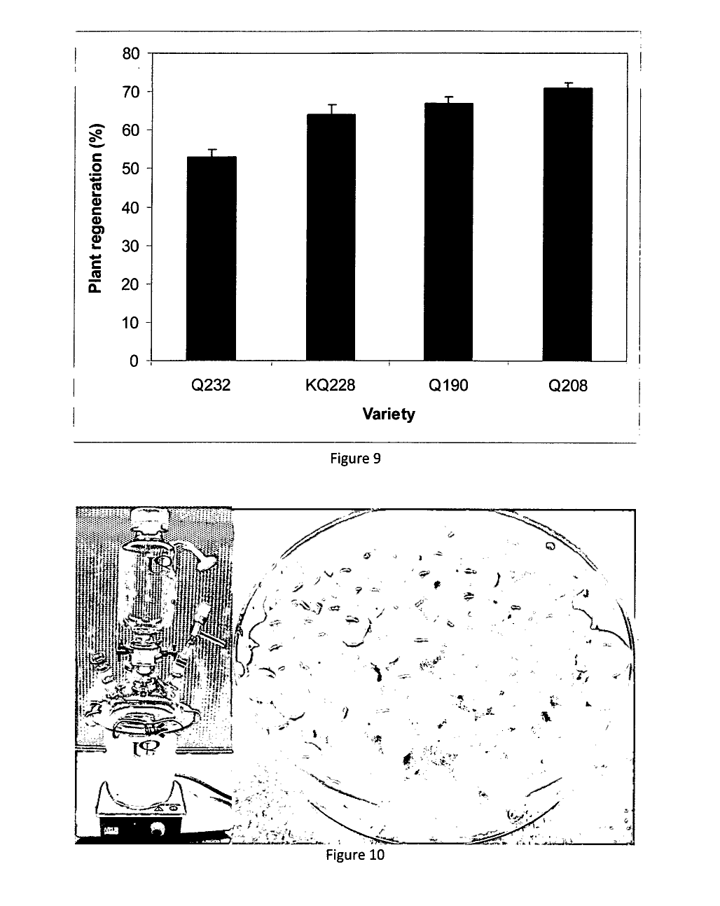

The artificial plant seed system developed for KQ228 has been adapted to other cultivars. This is one of the strengths of this technique in that it can work with different varieties of sugarcane. The difference between varieties is only seen in the subculture time. Some varieties require a longer pre-culture time on agar prior to encapsulation. There was a significant difference between the plant regeneration rate of varieties when all varieties had identical pr-culture periods although this is expected as regeneration is genotype dependant (FIG. 9). To minimize the decrease in regeneration of other varieties, an extra 1 or 2 weeks of culture on agar were included to increase the age of the bud and meristem tissue used for artificial plant seed production.

### Example 4

### Laboratory-Scale Apparatus for Artificial Plant Seed Production

A system for tissue encapsulation has been conceived and constructed (FIGS. 4 and 10). The machine has 2 chambers and requires a stirrer mechanism on the bottom. The lower chamber contains the calcium chloride solution (end a stirrer bar). The upper chamber contains the alginate and xanthan mix with the 4-week-old fragmented micro-shoot tissue. By slowly releasing the vacuum release valve on the top of the lower chamber droplets of alginate and tissue descend through a 9 mm orifice at the bottom of the upper chamber into the lower chamber. When the two liquids mix, the droplets set into a gel bead (ball shaped) containing tissue fragment. This is also known as an artificial plant seed. The artificial plant seeds remain in the bottom chamber stirring for 30 minutes. They are decanted off and rinsed twice in sterile deionised water and transferred to liquid medium for germination. The innovation of this concept is primarily in the efficiency area. Many artificial plant seeds can be made in a short timespan. Most artificial plant seed inventions rely on an operator picking up individual embryos of tissue pieces and placing them in the gelling solution. This solution covered fragment is then dropped by hand or pipette into the firming solution. This is a long and arduous process.

Whilst this machine is only for laboratory-scale amounts the concept has been proven and easily shows that artificial plant seed production can be performed efficiently. The encapsulation method incorporates a 3-4% w/v sodium alginate+1-1.5% w/v xanthan solution. When the alginate mix comes into contact with the cold, sterile 0.06 M CaCl₂ solution the alginate solution begins to harden (FIGS. 4 and 10).

Determining the concentration of sodium alginate and xanthan was critical for developing the encapsulation system using fragmented micro-shoots derived from axillary buds and shoot apex tissue. The density of the plant tissue was greater and so the tissue sank during encapsulation and blockages occurred. The amount of sodium alginate and xanthan was adjusted to 3% w/v sodium alginate+1% w/v xanthan (for 7 g tissue). This produced approx 375 artificial plant seeds/100 ml solution. Of these nearly 80% were useable and further improvements to this number are expected with the use of the plant tissue processing apparatus and the pilot-scale artificial plant seed production apparatus.

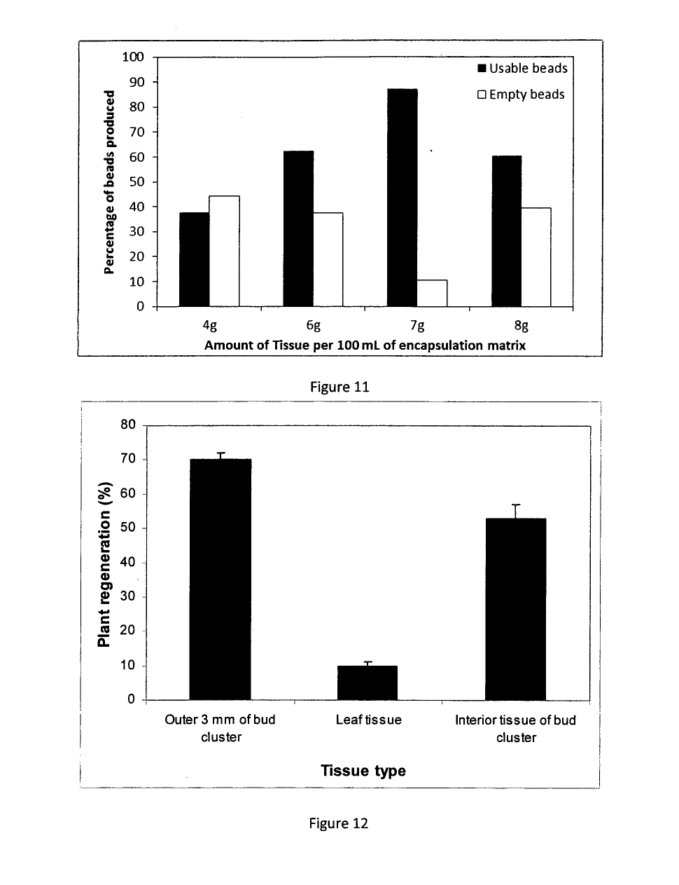

The ratio of tissue to alginate solution was also tested with 70 g of tissue/L. This ratio is important as it does not cause blockages in the encapsulation apparatus currently developed and there is a greater number of artificial plant seeds produced with the lowest number of empty artificial plant seeds (FIG. 11).

### Example 5

### Influence of Tissue-Type on the Germination of Artificial Plant Seeds

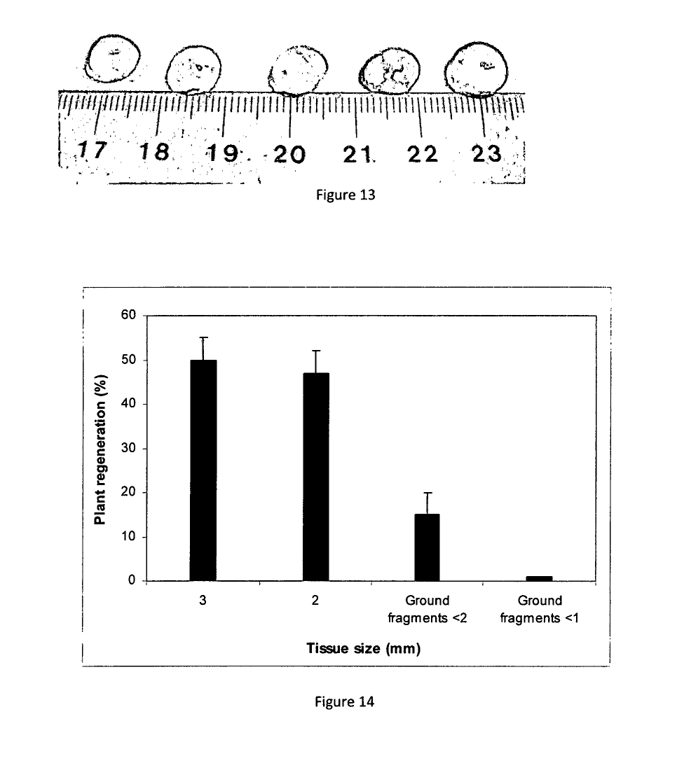

The artificial plant seeds are approximately 9-10 mm in size and am an oval-spherical shape (FIG. 13). The optimal seed size is determined by two variables: the minimum tissue fragment size needed for growth in the current culture condition and the mechanics of the laboratory-sale artificial plant seed production apparatus. Experiments with 2, 3 and 4 mm fragment slices showed the 3 mm slices to be the best for plant regeneration and the easiest to cut by hand (prior to the development of tissue processing apparatus). Two millimetre fragments were also effective for regeneration but it was difficult to accurately cut the tissue at 2 mm intervals without damaging the tissue (FIG. 14). Further improvements in tissue cutting may allow more efficient use of tissue size without any loss in regeneration efficiency.

The laboratory-scale plant seed production apparatus is another determinant of seed size. Because the apparatus relies on a vacuum to release the alginate/tissue mix into the calcium chloride and there is no stirrer mechanism in the upper chamber to keep the tissue and alginate mix homogeneous, the size of the orifice of the upper chamber where the plant tissue-coating solution and fragments drops from had to be optimised to achieve smooth and efficient production of useful artificial plant seeds.

### Example 6

### Plant Tissue Processing Apparatus for Fragmenting Tissues for the Production of Artificial Plant Seeds

A laboratory-scale sugarcane tissue dicer able to produce fragments of sugarcane tissue for encapsulation in an alginate matrix has been produced. Preliminary testing of this machine has proven successful with approximately equal sized fragments produced.

The machine is able to cut plant tissue without causing much damage and the tissue regenerates into plants. Tests for aseptic processing of tissue within the machine using standard laboratory procedures has been performed successfully. This included autoclaving the parts prior to use, as production of sterile tissue is critical as it will determine the operational practicalities of this machine for mass plant production. Tests with both autoclaved materials and sprays with 70% ethanol were successful and tissue contamination did not occur. This machine has shown that it is possible to develop a commercial scale system able to fragment plant material for artificial plant seed production.

### Example 7

### Field Trials

Field performance of various crops (SS: crops established with plants produced from leaf tissue (AU Patent 2001252043)), conventional micropropagated crops (MP; crops established with plants produced from axillary buds by traditional micropropagation) and artificial plant seed crops (AS; crops established with plants produced from artificial plant seeds from micro-shoot clusters) was compared with conventional one eye sett-propagated crops (OE; crops established with plants produced from one eye setts—the stem outtings from conventionally propagated field-grown plants) under commercial production conditions in two locations (Burdekin and Mackay).

Field trials for plantlets derived from artificial plant seeds have proven successful. Artificial plant seeds were produced and transferred to a nursery prior to planting in the field. This allowed the plantlets that emerged to harden off and establish stronger root system prior to planting in the field. Crops established with plantlets produced from artificial seeds (AS crop) was compared with SS, MP and OE crops. The artificial seed (AS) crop performed similar to others for all yield parameters assessed. For instance, there was no significant difference in cane and sugar yield between treatments (Table 1). A similar trend in crop performance was evident in Mackay as well but the trials showed large spatial variation.

### Example 8

### Integrated System for Production of Sugarcane Artificial Seeds

### Background

The main purpose of this work is to develop and implement advanced in vitro rapid propagation technologies for accelerated adoption of new conventionally developed as well as genetically modified sugarcane varieties. In vitro propagation technology, commonly referred to as micropropagation, is the most widely used plant biotechnology and is employed for large-scale production of high-value horticulture, floriculture and forestry plants worldwide. This is primarily done by propagating shoot meristem (an organogenically competent pre-existing tissue located in shoot apex and stem axils and capable of differentiating/developing into a complete plant in a permissive environment) and developing it into plantlets. This is a very labour intensive process, but a step change in productivity of propagation process in those crops where it is employed. The biggest advantage of shoot meristem-based conventional micropropagation is its ability to produce clonal (true-to-type) propagules more than any other in vitro propagation technologies (callus culture, cell culture, protoplast culture, direct organogenesis, somatic embryogenesis, etc). Micropropagation is largely practiced in low-cost countries. This conventional micropropagation technology is also applied for sugarcane propagation in many countries (e.g. Thailand, China, India, Brazil, and Indonesia). Currently the high cost and labour shortage are limiting its application in developed countries such as Australia.

### Traditional Commercial Sugarcane Propagation

There are two main methods for commercial sugarcane propagation:
1. Stick planting: as the name suggests, a new crop is raised by planting meter-long stem cuttings produced from whole stalk just prior to planting.
2. Billet planting: billets are smaller segments produced by cutting whole stalk into pieces with two intact nodes.
   Both methods are popular in Australia and in many other countries. About 770 million seedlings are needed to meet the annual planting material demand annually. In order to meet even a fraction of this demand requires a cost-effective highly efficient rapid propagation system. In order to achieve these outcomes an artificial seed system was developed.

### Development of Sugarcane Artificial Seed System

### What are the Specifications for Sugarcane Artificial Seed System?

1) Direct plantable seed-like propagules
2) True-to-type with a very low tolerance to off-types
3) Technology with high efficiency/productivity
4) Genotype independence
5) Opportunity to automate the entire or the majority of steps involved
6) Scalable technology
7) Cost-effectiveness
8) Capacity for off-season production, storage and transportation of propagules
9) Technology transferable to other crops

### Concepts and Technological Approaches Involved

### 1. Tissue Gardening and Production Off True-to-Type Propagule.

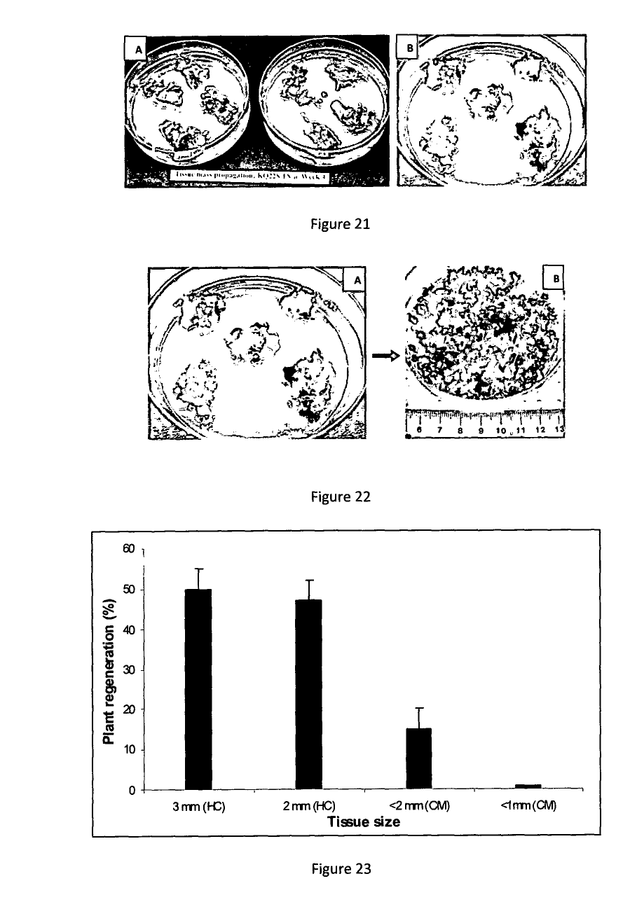

To produce genetically uniform propagules shoot and axillary meristems (from shoot tip and axillary buds, respectively) were used as the starting material. The first technical challenge was production of large quantities of organogenically competent meristematic tissue (tissue gardening) for artificial seed production (Table 1). Through experimentation a process for in vitro culture and proliferation of meristem without differentiating into shoots or plantlets was developed (FIG. 21).

This is achieved by breaking the apical dominance of shoot tip meristem allowing the axillaries to proliferate. Theoretically tissue proliferation can be continued indefinitely as long as the tissue remains meristematic. We routinely maintain meristematic tissue for 6 months for the production of artificial seeds. The key innovation hem is the ability to produce and maintain sugarcane meristematic tissue capable of regenerating into shoots or plantlets for extended periods under defined culture conditions.

### 2. Maximizing the Productivity of Tissue Gardening.

A key determinant of productivity of artificial seed technology is its ability to produce maximum number of plants from a minimum amount of tissue. This can be achieved by growing the proliferating meristematic tissue in a culture condition that permits plant regeneration (FIG. 22). However, another key requirement is to make the final product, “sugarcane artificial seed”, as small as possible for all practical purposes. These two requirements suggest that plants need to be regenerated from the smallest possible amount of meristematic tissue as fast as possible. To realize these objectives we have developed a culture system that can produce plants from meristematic tissue fragments as small as 2 mm (FIG. 23). The minimum size of tissue fragment with maximum productivity was found to be 3 mm (FIG. 23)

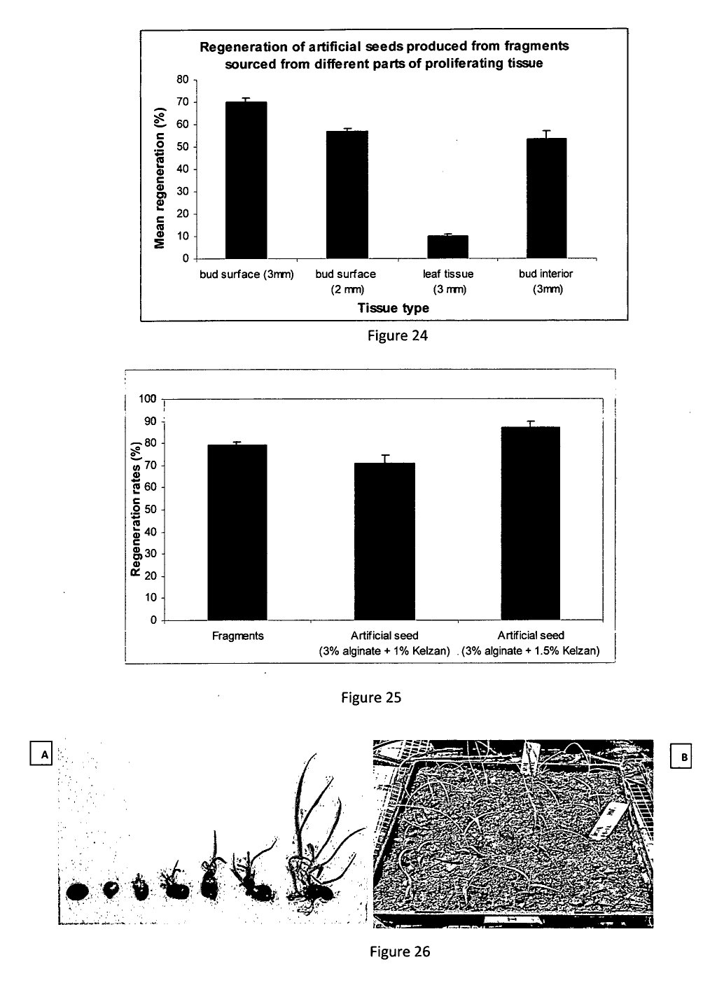

To further maximize the productivity of the system, experiments were conducted to identify the most regenerative part of the proliferating meristematic tissue mass (FIG. 24). Both 3 mm and 2 mm fragments were prepared from outer layer of tissue and the remaining inner core tissue. These two types of tissues were compared with 3 mm leaf whorl fragments for artificial seed production and subsequent plant regeneration. The results suggest that outer layer is more productive than the inner core with leaf whorl fragments the least regenerative.

The innovation in this step is that this way of producing sugarcane plants has not been demonstrated previously. In addition, successful production of plants in high frequency (80-90%) directly from small fragments, without intervening callus or somatic embryo production, forms a key technological advancement towards developing a potentially commercially viable artificial seed production system for sugarcane. Tissue fragment production at this stage was done manually, making the whole process labour-intensive.

3. Conversion of Tissue Fragments into a Seed-Like Structure Capable of Germinating into Plantlet:

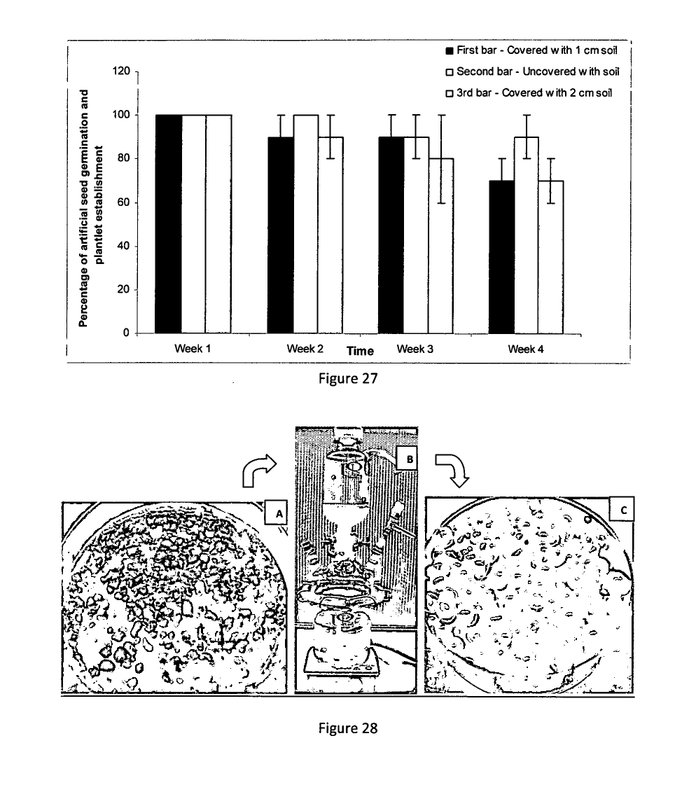

The next challenge was to determine whether tissue fragments can be made into a functionally seed-like structure (artificial seeds). This necessitated encapsulation of fragments in a biologically compatible matrix that carries moisture, nutrients, growth hormones, pesticides, etc to enable the seed to germinate and establish plantlet in soil. A suitable substrate for encapsulating fragments and a method for encapsulation has been established. The concept of artificial seeds is not new and has been experimentally demonstrated in many species. However, use of fragmented sugarcane meristems to increase productivity of artificial seed system and development of a suitable substrate (combination of sodium alginate and xanthan gum) are novel. Addition of xanthan gum was needed to achieve the required gel viscosity needed to adjust the production of seeds with single fragments (FIG. 25). With the optimized alginate-xanthan encapsulation matrix plant regeneration from artificial seeds has increased up to 85%. The artificial seeds germinated well (FIG. 26A) and produced normal plants in glasshouse trials (FIG. 26B). Nearly 80% of the artificial seeds sowed in soil germinated and developed into plantlets (FIG. 27).

### 4. Development of a Bench-Scale Immobilisation Apparatus for Rapid Production of Sugarcane Artificial Seeds:

A system for tissue immobilisation has been conceived and constructed (FIG. 28B). This machine is for bench-scale lab work and has been successfully used to produce artificial seeds. The machine has 2 chambers and requires a stirrer mechanism on the bottom. The lower chamber contains the calcium chloride solution (and a stirrer bar). The upper chamber contains the alginate and xanthan mix with tissue fragments (FIG. 28A). By slowly releasing the vacuum release valve on the top of the lower chamber droplets of alginate and tissue descend through a 9 mm orifice at the bottom of the upper chamber into the lower chamber. When the two liquids mix, the droplets set into a bead (ball shaped) containing tissue fragment. The beads (artificial seeds; FIG. 28C) remain in the bottom chamber stirring for 30 minutes. They are decanted off and rinsed twice in sterile deionised water and transferred to liquid medium for germination.

The immobilisation method incorporates a 3% w/v sodium alginate+1% w/v xanthan solution. When the alginate-tissue mix comes into contact with the cold, sterile 0.06 M CaCl₂ solution the alginate solution begins to harden and the artificial seed is formed. This technology is well established.

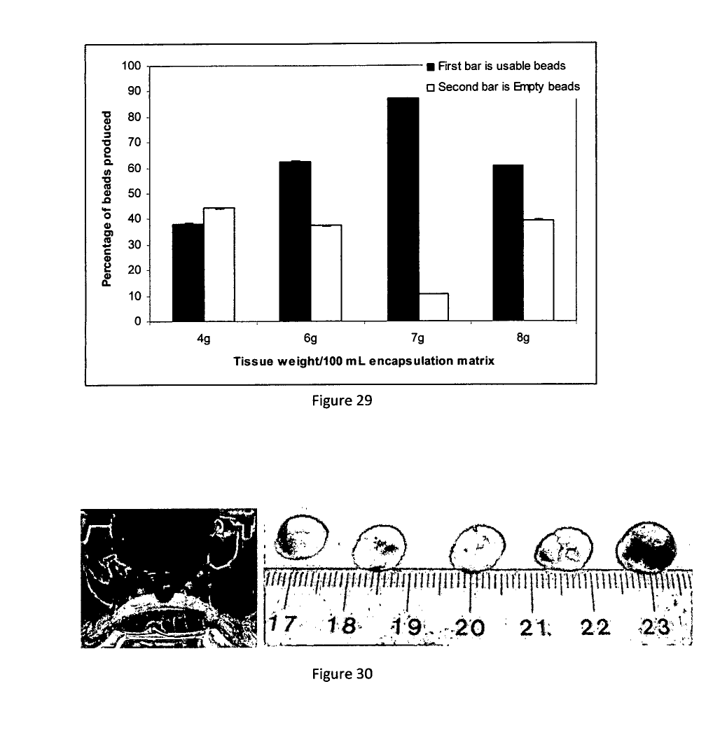

Determining the concentration of alginate and xanthan was critical for developing the immobilisation system using tissue fragments derived from axillary buds and shoot apex tissue. The density of the plant tissue was greater than the alginate solution and the tissue sank during immobilisation without proper beading. A 3% w/v sodium alginate and 1% w/v xanthan was found optimal for beading. This produced approx 375 beads/100 ml solution and nearly 80% of them germinated into plantlets (FIG. 27). The ratio of tissue to alginate solution was also tested with 70 g of tissue/L being the best for the immobilisation apparatus we are currently using (no blockages) and the highest number of beads produced with the lowest number of empty beads (FIG. 29).

### 5. Determining the Optimal Size of Artificial Seed:

The optimal seed size is determined by two critical variables: the minimum tissue fragment size needed for growth in the current culture condition and the mechanics of the bench-scale immobilisation machine. Experiments with different sizes of fragments showed the 3 mm fragment to be the best for plant regeneration and the easiest to out by hand (prior to the development of tissue processing machine) (FIG. 24). Two millimetre fragments were also effective for regeneration but it was difficult to accurately cut the tissue at 2 mm intervals without damaging the tissue.

The immobilisation apparatus (FIG. 28B) is the other determinant of seed size. Because the system we are using is relying on a vacuum to release alginate/tissue mix into calcium chloride solution and that there is no stirrer mechanism in the upper chamber to keep the tissue and alginate mix homogeneous, the size of the orifice of the upper chamber where the bead solution drops from had to be optimised to achieve smooth and efficient production of useful artificial seeds (FIG. 30A). The beads are approximately 9-10 mm in size and are an oval-spherical shape (FIG. 30B).

### 6. Automation of Tissue Fragmenting: Tissue Processing Machine (TPM):

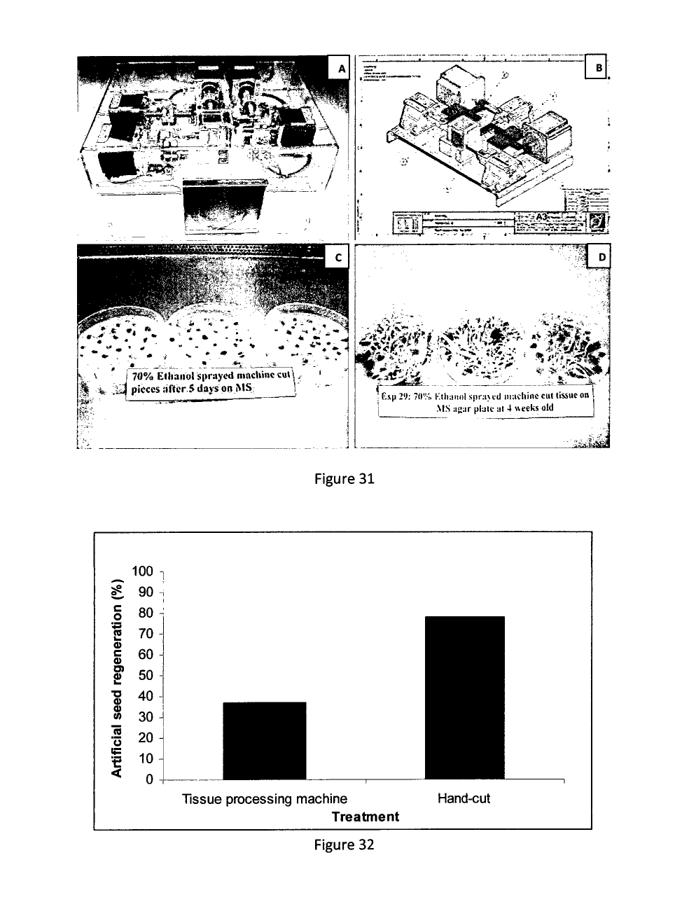

As mentioned earlier tissue fragmenting is a labour intensive process and without automating this step this technology will never become commercial. So a crucial part in achieving a commercial outcome to this work has been the development of a machine able to dice the tissue to the required specifications. A bench-top sugarcane tissue processing machine able to produce approximately equal sized fragments of sugarcane tissue for immobilisation in alginate beads has been developed and tested/used successfully (FIGS. 31, 32).

### 7. Application Artificial Seed Production System to Multiple Sugarcane Varieties:

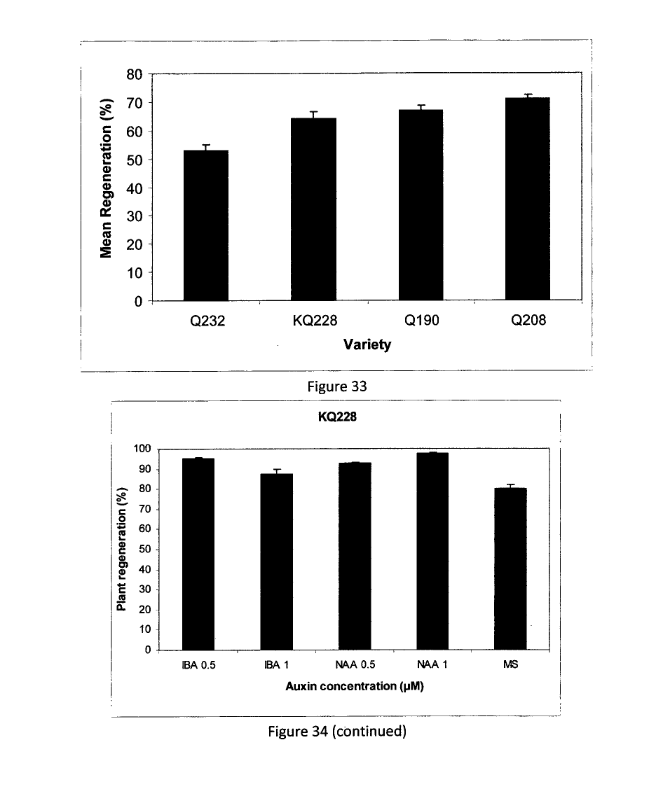

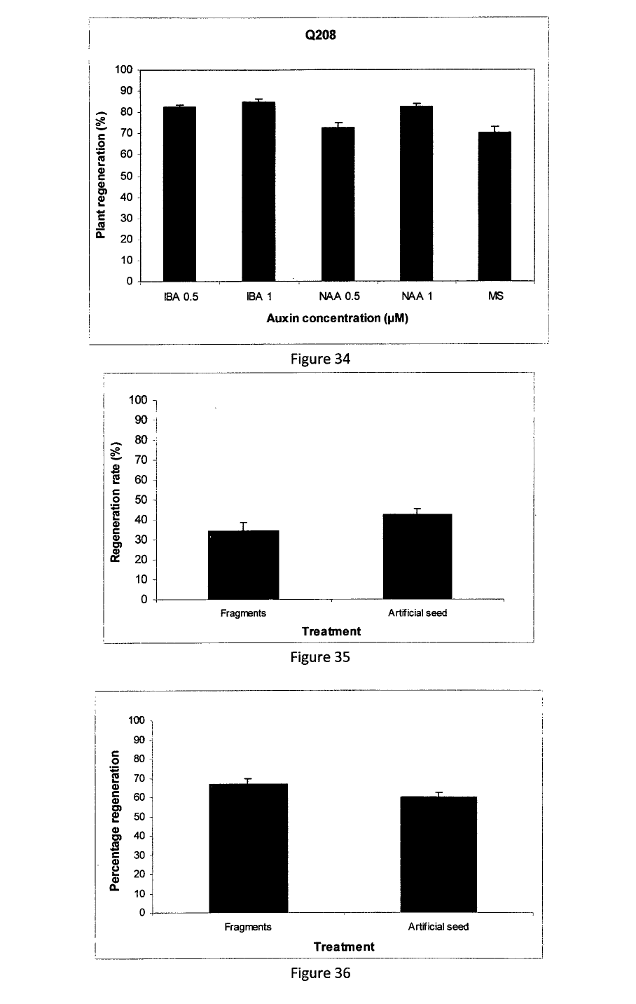

The artificial seed system developed for KQ228 has been adapted to other current cultivars. There was significant differences in germination rate between different varieties (FIG. 33). Most of the sugarcane artificial seeds produced both shoot and root system simultaneously when grown in vitro in liquid MS medium without any growth hormones (FIG. 33). Since artificial seeds were able to produce both shoot and root system simultaneously when sowed in soil they germinated and developed into plantlets. However, a significant number of artificial seeds developed into shoots with delayed rooting. The artificial seeds with delayed root formation tend to die in soil and to improve this situation, conversion of artificial seeds directly into plants were attempted in culture. Culturing sugarcane artificial seeds in MS medium supplemented with small amounts auxin indole-3 butyric acid (IBA) or α-naphthaleneacetic acid (NAA) improved conversion of seeds to plantlets (FIG. 34). This is largely due to auxin-induced improvement in rooting.

### Example 9

### Field Evaluation of Artificial Seeds

Field trials of commercial-scale crops established from artificial seeds of two most popular Australian commercial varieties, Q208 and KQ228 were conducted. In this trial artificial seed-derived crops were compared with conventionally propagated, micropropagated (by conventional in vitro technology) and Smartsett®-derived crops. The results indicate that propagation of sugarcane by artificial seed technology did not impact cane and sugar yield (Table 3), and ccs and fibre content (Table 4).

### Example 10

### Adaptation of Sugarcane Artificial Seed Technology to Other Crops: Banana and Ginger

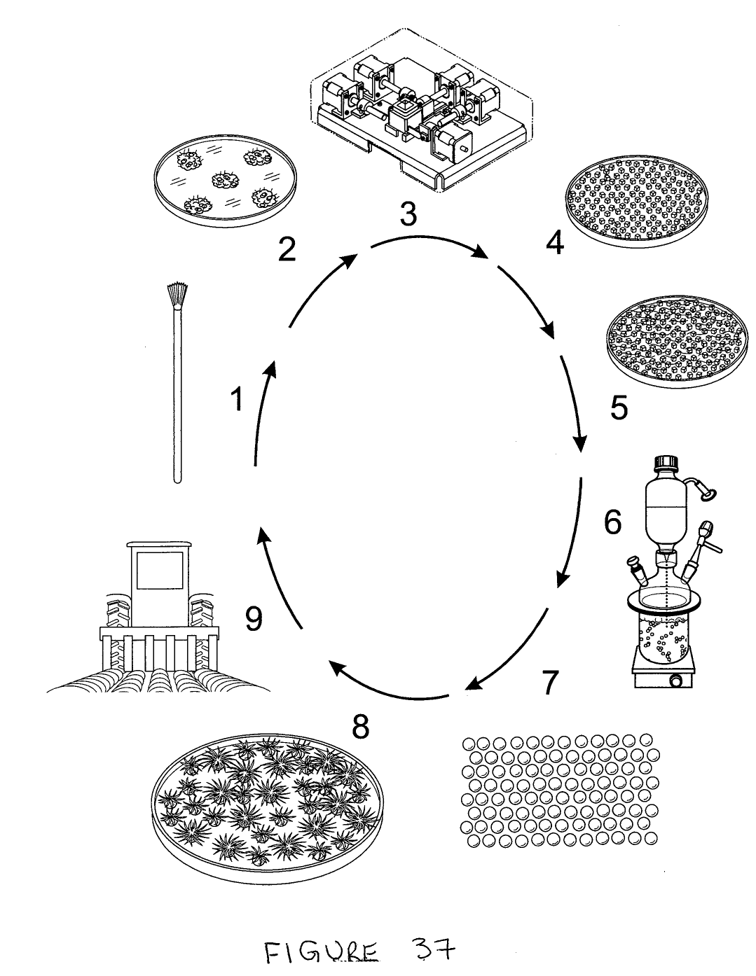

Here the inventors tested the application of sugarcane artificial seed technology in ginger and banana, two other monocot crops. The results show that the sugarcane artificial seed method as used in Example 8 can be used to propagate banana and ginger (FIGS. 35 and 36). The frequency of conversion of artificial seeds into plantlets in banana was low compared to ginger. This is not surprising considering the culture conditions optimized for sugarcane was used for these two crops. With further optimisation the efficiency of this system could be improved in these crops as well.

As observed in sugarcane no significant difference in plant regeneration between meristematic fragments and artificial seeds was recorded in both crops.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### TABLES

TABLE 1
Production of viable regenerative tissue from sugarcane leaf whorl or shoot tip for
preparation of artificial seeds. Commercial variety KQ228 was used for this experiment.

| | | | | | | Number |
|---|---|---|---|---|---|---|
| | | | | Culture | Average | of |
| | | Pre-RITA [symbol] | | duration | number of | artificial |
| Propagation | | culture | Treatment in | in RITA [symbol] | plants | seeds |
| method | Explant | conditions | RITA [symbol] | (week) | produced | produced |
| RITA [symbol] 1 | Leaf | Leaf whorls | 1 minute | 6 | 11 shoots per | NA |
| Temporary | whorls | were grown | tissue | | leaf whorl | |
| immersion | | on solid | immersion in | | | |
| system | | B4N10 in the | MS medium | | | |
| | | dark for 2 | every 48 hr | | | |
| | | weeks, then | 1 minute | 6 | 21 shoots per | NA |
| | | transferred | tissue | | leaf whorl | |
| | | to MS | immersion in | | | |
| | | medium³ for | MS medium | | | |
| | | 1 week under | containing | | | |
| | | 16 hr | 0.88 μM 6- | | | |
| | | photoperiod. | benzyladenine | | | |
| | | | every 48 hr | | | |
| RITA [symbol] 1 | Leaf | Leaf whorls | 1 minute | 7 | 41 shoots per | NA |
| Temporary | whorls | cultured for 2 | immersion | | leaf whorl | |
| immersion | | weeks on | every 24 hr in | | | |
| system | | B4N10⁽⁴⁾ in | MS medium | | | |
| | | the dark | | | | |
| | | Leaf whorls | 1 minute | 7 | 35 shoots per | NA |
| | | cultured for 2 | immersion | | leaf whorl | |
| | | weeks on | every 24 hr in | | | |
| | | B4N10 in the | MS medium | | | |
| | | dark then | | | | |
| | | transferred | | | | |
| | | to MS for 1 | | | | |
| | | week, 16 hr | | | | |
| | | light, 8 hr | | | | |
| | | dark | | | | |
| Shoot tip² | Shoot | Shoot tips or | 3 mm tissue | 4 | 63 shoots/shoot | 78/shoot |
| | tip or | axillary buds | fragments | | tip (using | tip |
| | axillary | were | coated in 3% | | artificial seeds) | |
| | bud | initiated on | alginate + | | | |
| | | B4⁽⁵⁾ solid | 1.5% Kelzan | | | |
| | | medium and | cultured in | | | |
| | | cultured for 4 | liquid MS | | | |
| | | weeks,. | medium on a | | | |
| | | Tissue grown | shaker, 16 hr | | | |
| | | during that | photoperiod | | | |
| | | period is | | | | |
| | | harvested | | | | |
| | | and cut into | | | | |
| | | 3 mm | | | | |
| | | fragments for | | | | |
| | | artificial seed | | | | |
| | | production | | | | |

¹Cultured leaf whorls were cut into 3 mm fragments. Fragments from 3 whorls were used for each RITA [symbol] unit.
²1.63 g tissue produced per shoot tip
³Murashige and Skoog medium
⁽⁴⁾B4N10 - MS medium supplemented with 4 μM 6-Benzylaminopurine and 10 μM naphthaleneacetic acid
⁽⁵⁾B4 - MS medium supplemented with 4 μM 6-Benzylaminopurine
NA = not applicable [symbol]
indicates data missing or illegible when filed

TABLE 2
Identifying the most appropriate encapsulation
matrix for artificial seed development

| Encapsulation matrix | | Matrix attributes | | |
|---|---|---|---|---|
| Sodium | | | | Optimal |
| Alginate* | Xanthan | Flow | | tissue |
| (% w/v) | (% w/v) | rate | Blockages | Suspension |
| 4% w/v | 0.5% w/v | Too fast | No | No |
| alginate | Kelgum | | | |
| 4% w/v | 0.5% w/v | Too fast | No | No |
| alginate | Keltrol | | | |
| 4% w/v | 0.5% w/v | Constant | No | No |
| alginate | Kelzan | | | |
| 4% w/v | 1.0% w/v | Constant | Rarely | Yes |
| alginate | Kelzan | | | |
| 4% w/v | 1.5% w/v | Too thick | Yes | Yes |
| alginate | Kelzan* | | | |

*Initial tests using 4% w/v alginate with a xanthan polymer were performed using SmartSett® leaf tissue fragment system. With the change to shoot and axillary meristem tissue later, the concentration of alginate was reduced to 3% w/v for optimal performance (refer to FIG. 25 as well).

TABLE 3
Cane and sugar yield of artificial seed-derived crop.
The data presented are of Plant Crop (first year crop).

| | | Treatments | | | | | |
|---|---|---|---|---|---|---|---|
| Trait | Clone | AS | SS | MP | OES | Factor | Lsd (5%) |
| TCH | KQ228 | 115 | 120 | 123 | 121 | Treatment | NS |
| | Q171 | | 117 | 115 | 102 | | |
| | Q200 | | 111 | 106 | 116 | | |
| | Q208 | 116 | 119 | 121 | 110 | | |
| TSH | KQ228 | 17.8 | 18.8 | 19.1 | 19.0 | Treatment | NS |
| | Q171 | | 16.4 | 16.8 | 15.0 | | |
| | Q200 | | 15.2 | 14.5 | 16.5 | | |
| | Q208 | 16.8 | 16.2 | 16.1 | 15.8 | | |

TCH = tonnes of cane/ha;
TSH = tonnes of sugar/ha;
AS, SS, MP and OES are crops established with planting material produced by artificial seeds, SmartSett ®, conventional micropropagation and one-eye-setts, respectively, from a conventionally propagated crop.

TABLE 4
Commercial cane sugar (ccs) and fibre content of artificial seed-derived
crop. The data presented are of Plant Crop (first year crop).
AS, SS, MP and OES are crops established with planting material
produced by artificial seeds, SmartSett ®, conventional
micropropagation and one-eye-setts, respectively, from a conventionally
propagated crop.

| | | Treatments | | | | | |
|---|---|---|---|---|---|---|---|
| Trait | Clone | AS | SS | MP | OES | Factor | Lsd (5%) |
| CCS | KQ228 | 15.4 | 15.8 | 15.6 | 15.8 | Treatment | NS |
| | Q171 | | 14.0 | 14.5 | 14.7 | | |
| | Q200 | | 13.6 | 13.6 | 15.0 | | |
| | Q208 | 14.5 | 13.8 | 13.4 | 13.8 | | |
| Fibre (%) | KQ228 | 14.6 | 15.0 | 14.7 | 14.2 | Treatment | NS |
| | Q171 | | 15.2 | 15.8 | 15.7 | | |
| | Q200 | | 16.1 | 15.7 | 16.2 | | |
| | Q208 | 16.0 | 16.3 | 16.1 | 15.4 | | |

1-90. (canceled)

91. A method of preparing a plant meristematic tissue fragment for use as a seed in plant micropropagation, said method including the steps of:
(i) inhibiting apical dominance of a plant meristematic tissue;
(ii) proliferating the plant meristematic tissue; and
(iii) fragmenting the plant meristematic tissue resulting from step (ii) to prepare the plant meristematic tissue fragment for use as a seed in plant micropropagation.

92. The method of claim 91, further comprising regenerating a plant or a plant tissue from the plant meristematic tissue fragment.

93. The method of claim 91, wherein steps (i) and/or (ii) further include culturing the plant meristematic tissue whilst maintaining inhibition of apical dominance.

94. The method of claim 91, wherein the plant meristematic tissue is cultured prior to inhibition of apical dominance.

95. The method of claim 91, wherein the plant meristematic tissue is cultured for between up to 1 week to up to 12 months.

96. The method of claim 91, wherein inhibiting apical dominance is by way of treatment selected from the group consisting of physical treatment, chemical treatment, biochemical treatment and environmental impact of the plant meristematic tissue.

97. The method of claim 96, wherein inhibiting apical dominance is by way of physical treatment.

98. The method of claim 97, wherein physical treatment is cutting the plant meristematic tissue.

99. The method of claim 98, wherein the plant meristematic tissue is cut along a longitudinal axis.

100. The method of claim 91, wherein the plant meristematic tissue is derived from shoot apex or axillary meristem.

101. The method of claim 91, wherein the plant meristematic tissue is of a monocotyledonous plant.

102. The method of claim 101, wherein the monocotyledonous plant is sugarcane.

103. The method of claim 101, wherein the monocotyledonous plant is banana.

104. The method of claim 91, wherein the plant meristematic tissue fragment has a mean size of between about 0.5 mm and about 20 mm.

105. The method of claim 91, wherein step (iii) is at least partially automated.

106. A method of producing an artificial plant seed, said method including the steps of:
(i) inhibiting apical dominance of a plant meristematic tissue;
(ii) proliferating the meristematic tissue;
(iii) fragmenting the plant meristematic tissue resulting from step (ii) to thereby produce a plant meristematic tissue fragment; and
(iv) coating the plant meristematic tissue fragment with a plant tissue-coating medium to thereby produce the artificial plant seed.

107. The method according to claim 106, wherein steps (i) and/or (ii) further include culturing the plant meristematic tissue whilst maintaining inhibition of apical dominance.

108. The method according to claim 106, wherein the plant meristematic tissue is cultured prior to inhibition of apical dominance.

109. The method according to claim 106, wherein inhibiting apical dominance is by way of treatment selected from the group consisting of physical treatment, chemical treatment, biochemical treatment and environmental impact of the plant meristematic tissue.

110. The method according to claim 109, wherein inhibiting apical dominance is by way of physical treatment.

111. The method according to claim 110, wherein physical treatment is cutting the plant meristematic tissue.

112. The method according to claim 111, wherein the plant meristematic tissue is cut along a longitudinal axis.

113. The method according to claim 106, wherein the plant meristematic tissue is derived from shoot apex or axillary meristem.

114. The method according to claim 106, wherein the plant meristematic tissue is of a monocotyledonous plant.

115. The method according to claim 114, wherein the monocotyledonous plant is sugarcane.

116. The method according to claim 114, wherein the monocotyledonous plant is banana.

117. The method according to claim 106, wherein the plant meristematic tissue fragment has a mean size of between about 0.5 mm and about 20 mm.

118. The method according to claim 106, wherein the plant tissue-coating medium comprises alginate and xanthan.

119. The method according to claim 106, wherein steps (iii) and/or (iv) are at least partially automated.
